(19) United States
(12) Patent Application Publication
(10) Pub. No.: US 2023/0230656 A1
(43) Pub. Date: Jul. 20, 2023

(54) COMPUTER-IMPLEMENTED METHOD FOR PROVIDING COVERAGE OF OLIGONUCLEOTIDE SET FOR PLURALITY OF NUCLEIC ACID SEQUENCES

(71) Applicant: SEEGENE, INC., Seoul (KR)

(72) Inventors: Hyun Ju RYOO, Seoul (KR); Do Hee KIM, Gyeonggi-do (KR); Dae Young KIM, Seoul (KR)

(21) Appl. No.: 18/011,758

(22) PCT Filed: Jun. 23, 2021
(86) PCT No.: PCT/KR2021/007912

§ 371 Date: Dec. 20, 2022

(30) Foreign Application Priority Data
Jun. 24, 2020 (KR) 10-2020-0077423

(51) Int. Cl. G16B 25/20; G16B 30/10

(52) U.S. Cl. CPC G16B 25/20; G16B 30/10

(57) ABSTRACT

The present invention relates to a computer-implemented method for providing a coverage of an oligonucleotide set for a plurality of nucleic acids. The present invention provides nucleic acid sequences with the generation of probe-hybridized amplicons and/or nucleic acid sequences without the generation of probe-hybridized amplicons, by a combination of oligonucleotides according to match or mismatch information and position information of a forward primer, a probe, and a reverse primer included in an oligonucleotide set, and thus can provide a coverage of the oligonucleotide set for a plurality of nucleic acid sequences, can analyze specificity of the oligonucleotide set, and can modify the sequences of the oligonucleotides included in the oligonucleotide set for the improvement in specificity. According to the present invention, the specificity analysis results can be compared between an oligonucleotide set of an existing product and an oligonucleotide set of a new product, and the specificity change of the oligonucleotide set can be easily monitored.

### CROSS-REFERENCE TO RELATED APPLICATIONS

This application claims priority from Korean Patent Application No. 2020-0077423, filed on Jun. 24, 2020 in the Korean Intellectual Property Office, the disclosure of which is incorporated herein by reference.

### BACKGROUND OF THE INVENTION

### Field of the Invention

The present invention relates to a computer-implemented method for providing a coverage of an oligonucleotide set for a plurality of nucleic acids.

### Description of the Related Art

The polymerase chain reaction (PCR), which is a nucleic acid amplification method, involves repeated cycles of denaturation of double-stranded DNA, oligonucleotide primer annealing to the DNA template, and primer extension by a DNA polymerase (Mullis et al., U.S. Pat. Nos. 4,683,195, 4,683,202, and 4,800,159; and Saiki et al., (1985) Science 230, 1350-1354).

PCR-based techniques have been widely used for amplification of target DNA sequences as well as scientific applications or methods in the fields of biological and medical research, such as reverse transcriptase PCR (RT-PCR), differential display PCR (DD-PCR), cloning of known or unknown genes by PCR, rapid amplification of cDNA ends (RACE), arbitrary priming PCR (AP-PCR), multiplex PCR, SNP genome typing, and PCR-based genomic analysis (McPherson and Moller (2000) PCR. BIOS Scientific Publishers, Springer-Verlag New York Berlin Heidelberg, N.Y.). Out of the PCR-based techniques, multiplex PCR means the simultaneous amplification and detection of multiple regions of one target nucleic acid molecule or a plurality of target nucleic acid molecules by using a combination of a plurality of oligonucleotide sets (forward and reverse primers, and probes) in one tube.

To provide an oligonucleotide set capable of amplifying and detecting a plurality of nucleic acid sequences of a particular target nucleic acid molecule with a maximum coverage, an oligonucleotide set having a performance capable of amplifying and detecting a plurality of nucleic acid sequences of a particular target nucleic acid molecule with a maximum coverage needs to be designed, and for testing the performance of the oligonucleotide set, the oligonucleotide set needs to be tested for specificity and sensitivity.

Before such performance tests, the designed oligonucleotide set needs to be confirmed for specificity, or after the oligonucleotide set is productized by passing through the performance tests, the productized oligonucleotide set needs to be further confirmed for specificity when new nucleic acid sequences for a particular target nucleic acid molecule are sequenced or the new nucleic acid sequences are registered in the database.

Conventional programs that have been generally used to confirm specificity of oligonucleotides are basic local alignment search tool (BLAST) and primer-BLAST algorithms accessible from the National Center for Biotechnology Information (NCBI).

In the BLAST(https://blast.ncbi.nlm.nih.gov/Blast.cgi) (Altschul et al., J. Mol. Biol. 215:403-10(1990)), when a nucleic acid sequence or oligonucleotide sequence to be analyzed is input as a query sequence, a nucleotide database containing nucleic acid sequences similar to sequences to be found is selected, and then nucleotide BLAST is performed, sequence information (accession numbers and the like) including sequences similar to the oligonucleotide sequence inputted as a query sequence, taxonomy, and alignment information of nucleic acid sequences hit as sequences similar to the oligonucleotide sequence are displayed.

However, in the BLAST, the analysis is performed in the unit of an oligonucleotide rather than an oligonucleotide set having a combination of a primer pair and a probe, and thus for analysis of specificity of an oligonucleotide set, a user needs to perform BLAST for each oligonucleotide included in the oligonucleotide set and combine specificity analysis results of primer pairs forming amplicons and probes hybridizing with the amplicons from the each BLAST result. However, a lot of nucleic acid sequences are contained in the nucleotide database, and thus it is practically difficult to analyze specificity of an oligonucleotide set forming amplicons by combining the analysis results of the oligonucleotides included in the oligonucleotide set.

In the primer-BLAST (https://www.ncbi.nlm.nih.gov/tools/primer-blast/index.cgi) (Ye et al., BMC Bioinformatics, 13:134(2012)), when one designed forward primer sequence and one designed reverse primer sequence are input as query sequences, a nucleotide database including nucleic acids to be amplified to the primer pair is selected, and then primer-BLAST is performed, sequence information (accession numbers) including sequences similar to the primer pair sequences inputted as query sequences and search summary information (the number of BLAST-hit sequences, the minimum value of mismatches, the maximum target size, and the like) are provided. However, the primer-BLAST has problems in that the match or mismatch patterns of the primer pair for all of the BLAST-hit sequences cannot be shown, the specificity analysis results of a probe to a product amplified by the primer pair cannot be known, and specificity analysis cannot be performed for a plurality of forward primers and/or reverse primers.

Therefore, the present inventors recognized the need to develop an algorithm capable of performing specificity analysis of an oligonucleotide set in the unit of a probe-hybridized amplicon, which is detected by hybridization of a probe with a product (that is, an amplicon) amplified by a forward primer and/or a reverse primer. Furthermore, specifically, the present inventors have attempted to develop an algorithm capable of performing specificity analysis even when at least one of forward primer, a probe, and a reverse probe included in an oligonucleotide set is plural in number.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have endeavored to develop a method capable of efficiently analyzing specificity of an oligonucleotide set (e.g., a primer pair and a probe) used to amplify and detect a plurality of target nucleic acid sequences. As a result, the present inventors verified that, unlike a conventional method of analyzing specificity to an oligonucleotide sequence or sequences of a primer pair, the present invention provides nucleic acid sequences with the generation of probe-hybridized amplicons and/or nucleic acid sequences without the generation of probe-hybridized amplicons, by a combination of oligonucleotides according to match or mismatch information and position information of a forward primer, a probe, and a reverse primer included in an oligonucleotide set, and thus can provide a coverage of the oligonucleotide set for a plurality of nucleic acid sequences, can analyze specificity of the oligonucleotide set, and can modify the sequences of the oligonucleotides included in the oligonucleotide set for the improvement in specificity, and therefore the present inventors completed the present invention.

Therefore, it is an object of the present invention to provide a computer-implemented method for providing a coverage of an oligonucleotide set for a plurality of nucleic acid sequences.

It is another object of the present invention to provide a computer-readable storage medium including instructions to implement a process to perform a method for providing a coverage of an oligonucleotide set for a plurality of target nucleic acid sequences.

Other objects and advantages of the present invention will become apparent from the detailed description to follow taken in conjugation with the appended claims and drawings.

### BRIEF DESCRIPTION OF THE DRAWINGS

FIG. 1 is a flowchart showing steps for performing a method of the present invention according to an embodiment.

FIG. 2 shows the results that oligonucleotide sets including forward primers (Fpri A-1 and Fpri A-2), probes (Probe B-1 and Probe B-2), and reverse primers (Rpri C-1 and Rpri C-2) generate probe-hybridized amplicons for a nucleic acid sequence. The match or mismatch information (match or mismatch types) and position information (position on the nucleic acid sequence) of each of the oligonucleotides included in the oligonucleotide sets are shown, and on the basis of these, combinations of oligonucleotides generating eight probe-hybridized amplicons are shown (lower part of FIG. 2).

FIG. 3 shows partial nucleotides and a template partial according to the presence or absence of a base in a region of a nucleic acid sequence, which is confirmed for a mismatch or match with an oligonucleotide.

FIG. 4 shows the results that an oligonucleotide set including the forward primer (Fpri A-1), probe (Probe B-1), and reverse primer (Rpri C-1) generates probe-hybridized amplicons for a nucleic acid sequence having partial nucleotides or a template partial. Amplicon Size represents the length of the probe-hybridized amplicon.

FIG. 5 shows that the mismatch patterns of oligonucleotide Nos. 1-1 and 1-2 are different from each other, and the mismatch patterns of oligonucleotide Nos. 2-1 and 2-2 are different from each other. Oligo represents the oligonucleotide, and Template represents the nucleic acid sequence.

FIG. 6 shows that when mismatch patterns of oligonucleotides are the same as each other while differing only in view of the presence or absence of partial nucleotides in templates (nucleic acid sequences), such mismatch patterns are the same mismatch pattern and thus are subjected to pattern merging. Oligo represents the oligonucleotide, and Template represents the nucleic acid sequence.

FIG. 7 shows the results of determining the probe-hybridized amplicon length. In FIG. 7, Acc. No, Fpri-1 and Fpri-2, Probe-1 and Probe-2, Rpri-1 and Rpri-2, and Amp. Size represent accession number, forward primers, probes, reverse primers and probe-hybridized amplicon length, respectively. XX01639.1 and the like expressed as accession numbers are arbitrarily described to explain the process of determining the probe-hybridized amplicon length.

FIG. 8 shows the results in Excel file format for some of 982 nucleic acid sequences producing probe-hybridized amplicon matches (probe-hybridized amplicon match nucleic acid sequences) by oligonucleotide set 1 for a nucleotide database containing nucleic acid sequences collected by the Taxonomy Trichophyton.

FIG. 9 shows the results in Excel file format for some of 988 nucleic acid sequences with the generation of probe-hybridized amplicon mismatches (probe-hybridized amplicon mismatch nucleic acid sequences) by oligonucleotide set 1 for a nucleotide database containing nucleic acid sequences collected under the Taxonomy Trichophyton.

FIG. 10 shows the results in Excel file format for some of 22786 nucleic acid sequences without the occurrence of probe-hybridized amplicons (probe-hybridized amplicon fail nucleic acid sequences) by oligonucleotide set 1 for a nucleotide database containing nucleic acid sequences collected under the Taxonomy Trichophyton.

FIGS. 11A and 11B show the information of the top two mismatch patterns with a larger number of nucleic acid sequences, among mismatch patterns of a combination of oligonucleotides generating probe-hybridized amplicons in oligonucleotide set 1. A single drawing is divided and shown in FIGS. 11A and 11B. In FIGS. 11A and 11B, #Acc represents the number of accession numbers; #TP represents the number of partial templates or template partials; FpriName represents the forward primer name; ProbeName represents the probe name; RpriName represents the reverse primer name; Fpri_MT, Probe_MT, and Rpri_MT represent match or mismatch types of the forward primer, probe, and reverse primer, respectively; Fpri_PT, Probe_PT, and Rpri_PT represent information of mismatch patterns of the forward primer, probe and reverse primer, respectively; TaxName represents the taxonomy name; and Acclist represents the accession number list.

### DETAILED DESCRIPTION OF THIS INVENTION

In an aspect of the present invention, there is provided a computer-implemented method for providing a coverage of an oligonucleotide set for a plurality of nucleic acid sequences, the method including:

(a) inputting sequences of an oligonucleotide set, wherein the oligonucleotide set includes a primer pair and a probe as oligonucleotides;

(b) providing a nucleotide database, wherein the nucleotide database contains a plurality of nucleic acid sequences;

(c) providing match or mismatch information and position information of each of the oligonucleotides included in the oligonucleotide set for each of the plurality of nucleic acid sequences by confirming whether the sequences of the oligonucleotide set are mismatched to the plurality of nucleic acid sequences contained in the nucleotide database, wherein the match or mismatch information indicates the number of matches or mismatches and/or a mismatch pattern of each of the oligonucleotides included in the oligonucleotide set for each of the plurality of nucleic acid sequences;

(d) confirming whether probe-hybridized amplicons are generated by the oligonucleotide set for each of the plurality of nucleic acid sequences, wherein the primer pair includes a forward primer and a reverse primer; the probe-hybridized amplicons are products amplified by the forward primer and/or reverse primer and indicate amplicons detected by hybridization of the probe included in the oligonucleotide set; and at least one of the probe-hybridized amplicons is formed by a combination of the oligonucleotides according to the match or mismatch information and position information of each of the oligonucleotides included in the oligonucleotide set for each of the plurality of nucleic acid sequences; and

(e) providing nucleic acid sequences with the generation of probe-hybridized amplicons by the oligonucleotide set and/or nucleic acid sequences without the generation of probe-hybridized amplicons by the oligonucleotide set, wherein the nucleic acid sequences with the generation of probe-hybridized amplicons are covered by the oligonucleotide set and the nucleic acid sequences without the generation of probe-hybridized amplicons are not covered by the oligonucleotide set.

As used herein, the term “coverage” refers to providing information on nucleic acid sequences hybridized or covered by a combination of forward and/or reverse primers, and a probe included in an oligonucleotide set. The information on the nucleic acid sequences may contain the number of nucleic acid sequences, accession numbers of nucleic acid sequences, taxonomy names to which the nucleic acid sequences belong, taxonomy IDs assigned to the taxonomy names, ratios of nucleic acid sequences hybridized or covered by a combination of oligonucleotides included in the oligonucleotide set relative to a total of nucleic acid sequences, and mismatch patterns of oligonucleotides included in the combination of the oligonucleotides.

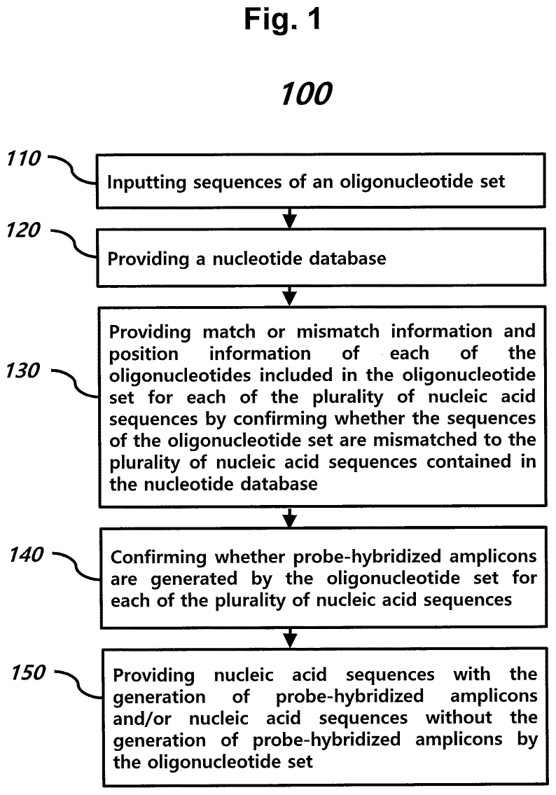

FIG. 1 is a flowchart showing steps for performing a method of the present invention according to an embodiment. The method of the present invention will be described with reference to FIG. 1 as follows.

### Step (a): Inputting Sequences of Oligonucleotide Set (110)

First, in the method of the present invention, sequences of an oligonucleotide set are input. The oligonucleotide set includes a primer pair and a probe as oligonucleotides.

The method of the present invention is a method that is implemented on a computer, and sequences of an oligonucleotide set to provide a coverage for a plurality of nucleic acid sequences are input to a user interface (UI).

The oligonucleotide set used in the present invention may be an oligonucleotide set that is designed to amplify and detect a plurality of nucleic acid sequences of a particular target nucleic acid molecule of a particular organism, or designed so and verified for performance.

The oligonucleotide set used in the present invention includes a primer pair and a probe as oligonucleotides. Specifically, the primer pair includes a forward primer and a reverse primer.

As used herein, the term “oligonucleotide” refers to a linear oligomer of natural or modified monomers or linkages. The oligonucleotide includes deoxyribonucleotides and ribonucleotides, can specifically hybridize with a target nucleotide sequence, and is naturally present or artificially synthesized. An oligonucleotide is especially a single chain for maximal efficiency in hybridization. Specifically, the oligonucleotide is an oligodeoxyribonucleotide. The oligonucleotide of the present invention may include naturally occurring dNMPs (i.e., dAMP, dGMP, dCMP and dTMP), nucleotide analogs, or derivatives. The oligonucleotide may also include a ribonucleotide. For example, the oligonucleotide used in the present invention may include nucleotides with backbone modifications, such as peptide nucleic acid (PNA) (M. Egholm et al., Nature, 365:566-568 (1993)), locked nucleic acid (LNA) (WO1999/014226), bridged nucleic acid (BNA) (WO2005/021570), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications, such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides with base modifications, such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine. Especially, the term “oligonucleotide” used herein is a single strand composed of a deoxyribonucleotide. The term “oligonucleotide” includes oligonucleotides that hybridize with cleavage fragments which occur depending on a target nucleic acid sequence.

As used herein, the term “primer” refers to an oligonucleotide that can act as a point of initiation of synthesis under conditions in which synthesis of primer extension products complementary to a target nucleic acid strand (a template) is induced, i.e., in the presence of nucleotides and a polymerase, such as DNA polymerase, and under appropriate temperature and pH conditions. The primer needs to be long enough to prime the synthesis of extension products in the presence of a polymerase. An appropriate length of the primer is determined according to a plurality of factors, including temperatures, fields of application, and primer sources.

As used herein, the term “probe” refers to a single-stranded nucleic acid molecule containing a portion or portions that are complementary to a target nucleic acid sequence. The probe may also contain a label capable of generating a signal for target detection.

The oligonucleotides may have typical primer and probe structures composed of a sequence hybridizing with a target nucleic acid sequence. Alternatively, the oligonucleotides may have distinctive structures through structural modification thereof. For example, the oligonucleotides may have a structure of Scorpion primer, Molecular beacon probe, Sunrise primer, HyBeacon probe, tagging probe, DPO primer or probe (WO 2006/095981), and PTO probe (WO 2012/096523).

The oligonucleotides may be modified oligonucleotides, such as a degenerate base-containing oligonucleotide and/or a universal base-containing oligonucleotide, in which degenerate bases and/or universal bases are introduced into a conventional primer or probe. As used herein, the terms “conventional primer”, “conventional probe”, and “conventional oligonucleotide” refer to a common primer, probe, and oligonucleotide, into which a degenerate base or non-natural base is not introduced. According to an embodiment, the degenerate base-containing oligonucleotide or universal base-containing oligonucleotide is an oligonucleotide of which at least 50%, at least 60%, at least 70%, at least 80%, at least 90% or at least 95% is not modified. According to an embodiment of the present invention, the number of degenerate bases or universal bases introduced into the conventional oligonucleotide is in the range of specifically 7 or less, 5 or less, 4 or less, 3 or less, or 2 or less. The use rate of the degenerate bases and/or universal bases introduced into the conventional oligonucleotide is specifically 25% or less, 20% or less, 18% or less, 16% or less, 14% or less, 12% or less, 10% or less, 8% or less, or 6% or less. The use proportion of the degenerate bases or universal bases represents a proportion of the degenerate bases or universal bases over a total of the nucleotides of the oligonucleotide into which the degenerate bases or universal bases are introduced. The degenerate bases include a variety of degenerate bases known in the art as follows: R: A or G; Y: C or T; S: G or C; W: A or T; K: G or T; M: A or C; B: C, G or T; D: A, G or T; H: A, C or T; V: A, C or G; and N: A, C, G or T. The universal bases include a variety of universal bases known in the art as follows: deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitropyrrole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, 2′-O-methoxyethyl 3-nitropyrrole, and a combination thereof. More specifically, the universal base is deoxyinosine, inosine, or a combination thereof.

According to an embodiment of the present invention, the primer included in the oligonucleotide set of the present invention is represented by Formula (I) below:

5′-X—Y—Z-3′ (I).

In the above formula, X represents a portion containing a hybridization nucleotide sequence to hybridize to a target nucleic acid sequence; Y represents a separation portion containing two or more consecutive bases not involved in Watson-Crick base pairing; and Z represents a portion containing a hybridization nucleotide sequence to hybridize to the target nucleic acid sequence.

The primer of Formula (I) has three different portions with distinctive properties, and its annealing specificity for the target nucleic acid sequence is doubly determined by their two separated portions, namely, portion X and portion Z.

In general, the annealing specificity of a conventional (typical) primer is dominated by its entire sequence. However, the annealing specificity of the primer of Formula (I) is doubly determined by two portions separated by portion Y, namely, portion X and portion Z.

Examples of the bases that are included in separation portion Y in Formula (I) and not involved in Watson-Crick base pairing include: (i) an unnatural base; (ii) a universal base; and (iii) a mismatched base.

As used herein, the term “non-natural base” refers to a derivative of a natural base, such as adenine (A), guanine (G), thymine (T), cytosine (C), and uracil (U), which are capable of forming a hydrogen-bonding base pair (see U.S. Pat. Nos. 8,440,406). Examples thereof include iso-C/iso-G, iso-dC/iso-dG, K/X, H/J, and M/N (see U.S. Pat. Nos. 7,422,850 and 8,440,406).

The term “universal base” refers to a base capable of forming a base pair with each of the natural DNA/RNA bases without discrimination, and the base pair does not participate in Watson-Crick base pairing. Examples of the universal base are as described above.

As used herein, the term “mismatched base” refers to a base incapable of forming a hydrogen bond base pair with an opposite base in a target nucleic acid sequence (see WO 2013/123552 and WO 2014/124290). The mismatched base may vary depending on the type of the opposite base in the target nucleic acid.

Portion Y may have two consecutive bases not involved in Watson-Crick base pairing, and specifically 3, 4, 5, 6, 7, or more consecutive bases not involved in Watson-Crick base pairing.

In the primer of Formula (I), portions X and Z each are a portion having a hybridization nucleotide sequence to a target nucleic acid sequence, that is, a portion having a hybridization nucleotide sequence complementary to a position on a template nucleic acid to hybridize therewith.

The portion X and/or the portion Z in the primer of Formula (I) may have one or more mismatches to a template (target nucleic acid sequence) to an extent that it can action as a primer. For example, the portion X and/or the portion Z in the primer of Formula (I) may have 1-2, 1-3, or 1-4 non-complementary nucleotides, and specifically, the portion X and/or the portion Z may have a nucleotide sequence that is perfectly complementary to one location on a template, that is, no mismatches.

The length of the portion X and the portion Z each may be in the range from 3 to 50 nucleotides.

In an embodiment, the portion X is longer than the portion Z. Specifically, the length of the portion X is 15 to 50, 15 to 40, 15 to 30, or 15 or 25 nucleotides, and the length of the portion Z is 3 to 15, 3 to 12, or 3 to 10 nucleotides.

In an embodiment, the portion Z is longer than the portion X. Specifically, the length of the portion Z is 15 to 50, 15 to 40, 15 to 30, or 15 or 25 nucleotides, and the length of the portion X is 3 to 15, 3 to 12, or 3 to 10 nucleotides.

In an embodiment, the Tm of the portions X and Z each is in the range of 6° C. to 80° C., 6° C. to 70° C., 6° C. to 60° C., 6° C. to 50° C., 6° C. to 40° C., 10° C. to 80° C., 10° C. to 70° C., 10° C. to 60° C., 10° C. to 50° C., 10° C. to 40° C., 20° C. to 80° C., 20° C. to 70° C., 20° C. to 60° C., 20° C. to 50° C., 20° C. to 40° C., 30° C. to 80° C., 30° C. to 70° C., 30° C. to 60° C., 30° C. to 50° C., or 30° C. to 40° C. In an embodiment, the Tm of the portion Y is in the range of 1° C. to 15° C., 1° C. to 20° C., 1° C. to 5° C., 2° C. to 15° C., 2° C. to 10° C., 2° C. to 5° C., 3° C. to 15° C., 3° C. to 10° C., or 3° C. to 5° C. In an embodiment, the Tm of the portion Y is lower than the Tm of the portions X and Z each.

In an embodiment, the Tm of the portion X is higher than the Tm of the portion Z. In a particular embodiment, the Tm of the portion X is 5° C., 10° C., 15° C., 20° C. or 25° C. higher than the Tm of the portion Z. In an embodiment, the Tm of the portion Z is higher than the Tm of the portion X. In a particular embodiment, the Tm of the portion Z is 5° C., 10° C., 15° C., 20° C. or 25° C. higher than the Tm of the portion X.

Specifically, the portion X herein may be expressed as a 5′-high Tm specificity portion, and the portion Z may be expressed as a 3′-low Tm specificity portion.

According to a particular embodiment of the present invention, the primer represented by Formula (I) is a dual specificity oligonucleotide (referred to as DSO or DPO) as disclosed in WO 2006/095981. For details with respect to the dual specificity oligonucleotide, refer to supra.

According to an embodiment of the present invention, the primer included in the oligonucleotide set of the present invention is a universal base primer (UBP). Specifically, the UBP has 1 to 3 universal base nucleotides; one or two of the universal base nucleotides are located in the core region ranging from the 3rd nucleotide to the 6th nucleotide at the 3′-end of UBP, and the rest are located in the range from the 4th nucleotide at the 5′-end of the UBP to the 7th nucleotide at the 3′-end of the UBP; and the universal base nucleotides are non-consecutive in UBP.

The term “universal base primer” as used herein refers to a primer in which at least one nucleotide in a primer contains a universal base instead of a naturally occurring base (A, C, G or T (U)). The UBP acts as an inhibitor against primer dimer formation. In particular, a primer containing deoxyinosine or inosine among universal bases is referred to as Inosine Primer (IPm).

In an embodiment, UBP has 1 to 2 universal base nucleotides.

The term “universal base nucleotide” refers to a nucleotide containing a universal base instead of a naturally occurring base. The above term may be used interchangeably with “universal nucleotide”, “universal base-containing nucleotide”, “universal base-including nucleotide”, or the like. Examples of the universal base are as described above.

The term “core region” as used herein refers to the optimal position range for locating one or two universal base nucleotides in UBP in order to attain the inhibition of primer dimer formation, particularly two-strand extendable primer dimer formation. That is, the core region refers to a specific region in the UBP where one or two universal base nucleotides are located in order to exert the maximum effect.

According to an embodiment of the present invention, the core region ranges from the 3rd nucleotide to 6th nucleotide, the 3rd nucleotide to 5th nucleotide, the 3rd nucleotide to 4th nucleotide, the 4th nucleotide to 6th nucleotide, the 4th nucleotide to 5th nucleotide, or the 5rd nucleotide to 6th nucleotide from the 3′-end of the UBP, and especially, the core region ranges from the 3rd nucleotide to 5th nucleotide from the 3′-end of the UBP.

As used herein, the expression “universal base nucleotides are nonconsecutive in the UBP” can be used interchangeably with the expression “the universal base nucleotides are located apart from each other in the UBP”. As used herein, the expression “universal base nucleotides are located apart from each other in the UBP” means that between the two universal base nucleotides, other nucleotide(s) are present. For example, the expression “universal base nucleotides are located two nucleotides apart from each other in the UBP” means that between the two universal base nucleotides, two other nucleotides are present.

In an embodiment of the present invention, the universal base nucleotides are located at least 1, 2, 3, 5, 8, 10, 12, 15 and 20 nucleotides apart from each other in the UBP.

In an embodiment of the present invention, the universal base nucleotides are located 1 to 10 nucleotides apart from each other in the UBP, for example 1 to 8 nucleotides, 1 to 6 nucleotides, 1 to 4 nucleotides, 2 to 10 nucleotides, 2 to 8 nucleotides, 2 to 6 nucleotides, 2 to 4 nucleotides, 3 to 10 nucleotides, 3 to 8 nucleotides, 3 to 6 nucleotides, or 3 to 4 nucleotides apart from each other in the UBP.

According to the present invention, other universal base nucleotide(s) except for the universal base nucleotide(s) located in the core region, if present, are located in a region ranging from 4th nucleotide from the 5′-end of the UBP to 7th nucleotide from the 3′-end of the UBP.

According to an embodiment, one or two of the universal base nucleotides are located in the core region ranging from the 3rd nucleotide to the 5th nucleotide at the 3′-end of UBP, and the rest are located in the range from the 4th nucleotide at the 5′-end of the UBP to the 6th nucleotide at the 3′-end of the UBP.

For details with respect to the universal base primer, refer to the disclosure of WO 2006/095981.

According to an embodiment of the present invention, the probe included in the oligonucleotide set used in the present invention is a labeled probe specifically hybridizing with a target nucleic acid. The probe is used in a method of providing a signal when it hybridizes with a target nucleic acid sequence or when it is hybridized and cleaved. Examples of such a signal providing method include the molecular beacon method using a dual-labeled probe forming a hair-pin structure (Tyagi et al, Nature Biotechnology v. 14 Mar. 1996), the hybridization probe method using two probes single-labeled with a donor or an acceptor (Bernad et al, 147-148 Clin Chem 2000; 46), the Lux method using a single-labeled oligonucleotide (U.S. Pat. No. 7,537,886), and the TaqMan method using a cleavage reaction of double-labeled probes by 5′-nuclease activity of DNA polymerase as well as the hybridization of a dual-labeled probe (U.S. Pat. Nos. 5,210,015 and 5,538,848), but are not limited thereto.

According to an embodiment of the present invention, the probe included in the oligonucleotide set used in the present invention is a tagging probe including a targeting portion containing a hybridizing-complementary nucleotide sequence to the target nucleic acid sequence and a tagging portion containing a non-hybridizing-non-complementary nucleotide sequence to the target nucleic acid sequence. The tagging probe acts as a mediation oligonucleotide, and is used in a method of providing a signal by a duplex, which is formed in a manner dependent on the cleavage thereof, that is, formed depending on the presence of the target nucleic acid sequence. An example of such a method is the PTO cleavage and extension (PTOCE) method disclosed in WO 2012/096523, which is incorporated herein by reference.

As used herein, the term “target nucleic acid molecule”, “target molecule”, or “target nucleic acid” refers to a nucleotide molecule in an organism to be detected. A target nucleic acid molecule is generally given a particular name, and includes the whole genome and all nucleotide molecules constituting the genome (e.g., genes, pseudogenes, non-coding sequence molecules, untranslated regions, and some regions of the genome). A target nucleic acid molecule includes, for example, nucleic acids of the organism.

As used herein, the term “organism” refers to an organism which belongs to one genus, species, subspecies, subtype, genotype, serotype, strain, isolate, or cultivar. Examples of the organism include prokaryotic cells (e.g., Mycoplasma pneumoniae, Chlamydophlla pneumoniae, Legionella pneumophila, Haemophilus influenzae, Streptococcus pneumoniae, Bordetella pertussis, Bordetella parapertussis, Neisseria meningitidis, Listeria monocytogenes, Streptococcus agalactiae, Campylobacter, Clostridium difficile, Clostridium perfringens, Salmonella, Escherichia coli, Shigella, Vibrio, Yersinia enterocolitica, Aeromonas, Chlamydia trachomatis, Neisseria gonorrhoeae, Trichomonas vaginalis, Mycoplasma hominis, Mycoplasma genitalium, Ureaplasma urealyticum, Ureaplasma parvum, Mycobacterium tuberculosis), eukaryotic cells (e.g., protozoa and parasites, fungi, yeast, higher plants, lower animals, and higher animals including mammals and humans), viruses, or viroids. Examples of the parasites in the prokaryotic cells include Giardia lamblia, Entamoeba histolytica, Cryptosporidium, Blastocystis hominis, Dientamoeba fragilis, and Cyclospora cayetanensis. Examples of the viruses include: influenza A virus (Flu A), influenza B virus (Flu B), respiratory syncytial virus A (RSV A), respiratory syncytial virus B (RSV B), parainfluenza virus 1 (PIV 1), parainfluenza virus 2 (PIV 2), parainfluenza virus 3 (PIV 3), parainfluenza virus 4 (PIV 4), metapneumovirus (MPV), Human enterovirus (HEV), human bocavirus (HBoV), human rhinovirus (HRV), coronavirus (e.g., CoV NL63, CoV 229E, CoV OC43, CoV HKU1, SARS-CoV, MERS-CoV, or SARS-CoV-2), and adenovirus, which cause respiratory diseases, and specifically, coronavirus, and more specifically, SARS-CoV-2. Examples of the viruses also include norovirus, rotavirus, adenovirus, astrovirus, and sapovirus, which cause gastrointestinal diseases. Examples of the viruses also include human papillomavirus (HPV), middle east respiratory syndrome-related coronavirus (MERS-CoV), dengue virus, herpes simplex virus (HSV), human herpes virus (HHV), Epstein-Barr virus (EMV), varicella zoster virus (VZV), cytomegalovirus (CMV), HIV, hepatitis virus, and poliovirus.

As used herein, the term “target nucleic acid sequence” or “target sequence” refers to a particular target nucleic acid sequence representing a target nucleic acid molecule.

One target nucleic acid molecule, for example, one target gene, may have a particular target nucleic acid sequence; otherwise as for a target nucleic acid molecule exhibiting genetic diversity or genetic variability, the target nucleic acid molecule may have a plurality of target nucleic acid sequences with diversity. The plurality of target nucleic acid sequences in the present invention are target nucleic acid sequences with sequence similarity. Specifically, the target nucleic acid sequences with sequence similarity may be a plurality of target nucleic acid sequences of one target nucleic acid molecule or a plurality of target nucleic acid sequences of two or more target nucleic acid molecules.

In order to design oligonucleotides to amplify and detect a particular target nucleic acid molecule of a specific organism, a variety of methods known in the art may be performed. For example, an oligonucleotide set having a primer pair and a probe combined can be provided therein by collecting and aligning a plurality of target nucleic acid sequences for a particular target nucleic acid molecule, designing oligonucleotides to satisfy the design requirements for each of the plurality of target nucleic acid sequences, and then combining oligonucleotides without interferences therebetween.

The designed oligonucleotide set includes a probe designed to satisfy at least one of the following requirements: (i) a Tm value of 50-85° C.; (ii) a length of 15-50 nucleotides; (iii) the exclusion of a mononucleotide (G)ₙ run sequence in which n is at least 3; (iv) G or C at the 5′-end; and (v) a GC content of 40% or more at the 5′-end portion.

The probe design requirements include more specifically at least two, still more specifically at least three, still more specifically at least four, and still more specifically five of the above-described requirements.

Tm value among the design requirements is, for example, 50-80° C., 50-75° C., 55-80° C., 55-75° C., 60-80° C., 60-75° C., 65-80° C., or 60-75° C. Specifically, the Tm value among the design requirements is, for example, 55-80° C., 60-78° C., 63-78° C., 65-75° C., 67-75° C., or 65-73° C.

The length among the design conditions is, for example, 10 to 60 nucleotides, 10 to 50 nucleotides, 10 to 45 nucleotides, 10 to 40 nucleotides, 10 to 35 nucleotides, 15 to 60 nucleotides, 15 to 50 nucleotides, 15 to 45 nucleotides, 15 to 40 nucleotides, or 15 to 35 nucleotides.

An example among the design requirements is the exclusion of a mononucleotide (G)ₙ run sequence in which n is at least 3 or 4.

The GC content at the 5′-end portion of the probe is 40% or more, specifically, 40-70%, or 40-60%. The 5′-end portion means a region within 10 nucleotides from the 5′-end of the probe.

The designed oligonucleotide set includes a probe designed to satisfy at least one of the following requirements: (i) the Tm value of the targeting portion being 50-85° C.; (ii) the length of the targeting portion being 15-50 nucleotides; (iii) three or more G-run sequences in the targeting portion being excluded; (iv) G or C at the 5′-end of the targeting portion; (v) the GC content at the 5′-end portion of the targeting portion is 40% or more; (vi) the length of the tagging portion being 6-30 nucleotides; (vii) 30% or more of mismatch sequences being included with respect to the length of the tagging portion; and (viii) 40% or more of mismatch sequences being included with respect to the length of the 3′-end portion of the tagging portion.

Among the design requirements for the targeting portion of the tagging probe, the Tm value, length, exclusion of a G-run sequence, G or C at the 5′-end, and the GC content at the 5′-end portion may be described with reference to the explanation of the general(conventional) probe.

The length of the tagging portion is specifically 6-20 nucleotides, 10-30 nucleotides, 10-20 nucleotides, 12-30 nucleotides, or 12-20 nucleotides.

Because the tagging portion has sufficient non-complementarity with respect to a certain region of the nucleic acid sequence to which the tagging probe hybridizes, it should not hybridize to the certain region under conditions in which the targeting portion of the tagging probe is hybridized. The tagging portion includes a mismatching sequence of specifically 40% or more, more specifically 50% or more of the length thereof. Specifically, the 3′-end portion of the tagging portion includes a mismatching sequence of 50% or more of the length thereof.

The designed oligonucleotide set includes a primer designed to satisfy at least one of the following requirements: (i) a Tm value of 40-70° C.; (ii) a length of 15-60 nucleotides; and (iii) the exclusion of a mononucleotide (G)ₙ run sequence in which n is at least 3.

The Tm value among the design requirements is, for example, 40-70° C., 50-70° C., 55-70° C., 45-65° C., 50-65° C., 55-65° C., 45-60° C., or 50-75° C. Specifically, the Tm value among the design requirements is, for example, 40-70° C., 45-65° C., 50-65° C., 50-60° C., 55-65° C., or 55-60° C.

The length among the design requirements is, for example, 15 to 60 nucleotides, 15 to 50 nucleotides, 15 to 45 nucleotides, 15 to 40 nucleotides, 15 to 35 nucleotides, 15 to 30 nucleotides, 15 to 25 nucleotides, 18 to 45 nucleotides, 18 to 40 nucleotides, 18 to 35 nucleotides, 18 to 30 nucleotides, or 18 to 25 nucleotides. Specifically, the length among the design requirements is, for example, 15 to 40 nucleotides, 16 to 40 nucleotides, 17 to 40 nucleotides, 18 to 40 nucleotides, 15 to 35 nucleotides, 16 to 35 nucleotides, 17 to 35 nucleotides, 18 to 35 nucleotides, 15 to 30 nucleotides, 16 to 30 nucleotides, 17 to 30 nucleotides, 18 to 30 nucleotides, 18 to 25 nucleotides, or 17 to 25 nucleotides.

The mononucleotide (G)ₙ run sequence among the design requirements has a criterion, for example, a mononucleotide (G)ₙ run sequence in which n is at least 3 or 4 being excluded.

In cases where the primer is a DPO primer developed by the present applicant (see U.S. Pat. No. 8,092,997), the descriptions for the Tm and the length of the DPO primer disclosed in the patent document may be presented as the design requirements.

The design requirements for a primer include more specifically at least two, and still more specifically at least three of the above-described requirements.

According to an embodiment of the present invention, the oligonucleotide set in step (a) further includes at least one oligonucleotide selected from the oligonucleotides consisting of at least one forward primer, at least one probe, and at least one reverse primer. For example, the oligonucleotides included in the oligonucleotide set may have two or more forward primers, two or more probes, or two or more reverse primers.

The use of an oligonucleotide set having two or more oligonucleotides of any one type of a forward primer, a probe and a reverse primer is advantageous with respect to the diagnosis of pathogens having genomes exhibiting genetic diversity or genetic variability.

In particular, genetic diversity is most frequently found and occurs in viral genomes (Bastien N. et al., Journal of Clinical Microbiology, 42:3532(2004); Peret TC. et al., Journal of Infectious Diseases, 185:1660(2002); Ebihara T. et al., Journal of Clinical Microbiology, 42:126(2004); Jenny-Avital ER. et al. Clinical Infectious Diseases, 32:1227(2001); Duffy S. et. al., Nat. Rev. Genet. 9(4):267-76(2008); Tong Y G et. al., Nature. 22:526(2015)).

According to an embodiment of the present invention, the method further includes, after step (a), step a-1) inputting sequences of at least one oligonucleotide set, which are different from the sequences of the oligonucleotide set in step (a).

According to an embodiment of the present invention, the coverage of each of a plurality of oligonucleotide sets can be analyzed by inputting sequences of the plurality of oligonucleotide sets.

More specifically, the at least one different oligonucleotide set in step a-1) may be the same as or different from the oligonucleotide set in step (a) in view of a nucleic acid molecule or organism to be covered. In addition, the sequence of at least one oligonucleotide of the oligonucleotides included in the at least one different oligonucleotide set in step a-1) may be different from the sequences of the oligonucleotides included in the oligonucleotide set in step (a).

When the sequences of the plurality of oligonucleotide sets are inputted in steps (a) and a-1), the plurality of oligonucleotide sets are subjected to steps (b) to (e) to be later described.

### Step (b): Providing Nucleotide Database (120)

Then, the method of the present invention provides a nucleotide database. The nucleotide database contains a plurality of nucleic acid sequences.

According to an embodiment of the present invention, the nucleotide database in step (b) is a nucleotide database containing nucleic acid sequences collected by an identifier selected from identifiers composed of taxonomy ID, taxonomy name, organism name, and target nucleic acid molecule name, from a public-accessible nucleotide database or a nucleotide database obtaining by downloading the public-accessible nucleotide database, or a nucleotide database containing nucleic acid sequences collected by a user. More specifically, the public-accessible nucleotide database is a nucleotide database selected from the group consisting of GenBank, European Molecular Biology Laboratory (EMBL), and DNA DataBank of Japan (DDBJ).

For a nucleotide database for collection of nucleic acid sequences, a public-accessible nucleotide database itself may be used, or a nucleotide database obtained by downloading the public-accessible nucleotide database may be used. The use of the downloaded nucleotide database enables the stable collection of nucleic acid sequences.

All the plurality of nucleic acid sequences included in the public-accessible nucleotide database or the database obtained by downloading the public-accessible nucleotide database may be used, and specifically, a nucleotide database containing nucleic acid sequences collected under an identifier selected from identifiers composed of taxonomy ID, taxonomy name, organism name, and target nucleic acid molecule name, from the public-accessible nucleotide database or the nucleotide database obtaining by downloading the public-accessible nucleotide database may be used. Alternatively, a nucleotide database containing nucleic acid sequences that are not listed in a public-accessible nucleotide database, that is, nucleic acid sequences collected by a user, may also be used.

According to an embodiment, a nucleotide database containing nucleic acid sequences collected by a particular identifier is used. For example, when a nucleotide database containing nucleic acid sequences collected through taxonomy ID and/or taxonomy name is provided, a nucleotide database containing not only nucleic acid sequences including information on taxonomy IDs and/or taxonomy names but also nucleic acid sequences with respect to taxonomy IDs and/or taxonomy names classified as subclasses of the taxonomy IDs and/or taxonomy names is provided. Therefore, the speed and accuracy of analysis can be improved by providing a nucleotide database containing nucleic acid sequences to be analyzed for a coverage, instead of all the nucleic acid sequences listed in a public-accessible nucleotide database.

According to an embodiment of the present invention, the oligonucleotide set in step (a) is the same as the nucleotide database in step (b) in view of information on an organism. The information on the organism indicates taxonomy ID, taxonomy name, or organism name, and herein, the meaning that the oligonucleotide set in step (a) is the same as the nucleotide database in step (b) in view of information on an organism is that the information on an organism, for which is to be amplified and detected by the designed oligonucleotide set in step (a), is the same as the information on an organism with respect to the plurality of nucleic acid sequences collected in order to provide the nucleotide database in step (b).

According to an embodiment of the present invention, the plurality of nucleic acid sequences include a plurality of target nucleic acid sequences and/or a plurality of non-target nucleic acid sequences.

The plurality of target nucleic acid sequences are at least 3, at least 5, at least 10, at least 20, at least 30, at least 40, at least 50, at least 60, at least 70, at least 80, at least 90, or at least 100 nucleic acid sequences.

As used herein, the term “target nucleic acid sequence” or “target sequence” refers to a particular nucleic acid sequence containing a target nucleic acid molecule to be amplified and detected by the oligonucleotide set.

The plurality of target nucleic acid sequences in the present invention are target nucleic acid sequences with sequence similarity. Specifically, the target nucleic acid sequences with sequence similarity may be a plurality of target nucleic acid sequences of one target nucleic acid molecule or a plurality of target nucleic acid sequences of two or more target nucleic acid molecules.

According to an embodiment of the present invention, the plurality of target nucleic acid sequences in the present invention are a plurality of nucleic acid sequences with sequence similarity for one target nucleic acid molecule with genetic diversity.

For example, the plurality of target nucleic acid sequences used in the present invention are a plurality of nucleic acid sequences having sequence similarity for a target nucleic acid molecule that exhibits genetic diversity, such as a viral genome sequence. For example, when an influenza A virus is to be detected and the M gene is determined as a target nucleic acid molecule, target nucleic acid sequences with diversity of the M gene of the influenza A virus may be used.

More specifically, the plurality of target nucleic acid sequences are a plurality of nucleic acid sequences of a whole genome sequence, a partial genome sequence, or one gene in viruses or bacteria, which have genetic diversity.

As used herein, the term “non-target nucleic acid sequence” or “non-target sequence” refers to a particular nucleic acid sequence containing a non-target nucleic acid molecule to be not amplified and detected by the oligonucleotide set.

As used herein, the term “non-target nucleic acid molecule” has a contrary concept to the above-described target nucleic acid molecule, and refers to a nucleic acid molecule that should not be detected in the detection procedure of a target nucleic acid molecule regardless of the homology with the sequence of the target nucleic acid molecule, and nucleic acid sequences of the non-target nucleic acid molecule may be used interchangeably with exclusive nucleic acid sequences.

According to an embodiment, the non-target nucleic acid molecule may be a molecule other than a target nucleic acid molecule. Alternatively, the non-target nucleic acid molecule may be selected. According to an embodiment, the non-target nucleic acid sequence may be a nucleic acid sequence other than target nucleic acid sequences. Alternatively, the non-target nucleic acid sequence may be selected.

According to the present embodiment, the coverage of the oligonucleotide set for a target nucleic acid sequence and/or the coverage of the oligonucleotide set for a non-target nucleic acid sequence may be analyzed.

According to an embodiment of the present invention, the target nucleic acid sequence or the non-target nucleic acid sequence is a genomic sequence containing a target nucleic acid molecule or non-target nucleic acid molecules. The present invention has a significant meaning in analyzing the coverage of an oligonucleotide set designed on the basis of a target nucleic acid molecule for the plurality of nucleic acid sequences, and thus the nucleic acid sequences indicate sequences containing sequences corresponding to the target nucleic acid molecule or the non-target nucleic acid molecule.

### Step (c): Providing Match or Mismatch Information and Position Information of Oligonucleotides Included in Oligonucleotide Set (130)

In the method of the present invention, match or mismatch information and position information of each of the oligonucleotides included in the oligonucleotide set for each of the plurality of nucleic acid sequences are provided by confirming whether the sequences of the oligonucleotide set are matched or mismatched to the plurality of nucleic acid sequences contained in the nucleotide database. The match or mismatch information indicates the number of matches or mismatches and/or a mismatch pattern of each of the oligonucleotides included in the oligonucleotide set to each of the plurality of nucleic acid sequences.

In an embodiment of the present invention, the position information indicates positions of the oligonucleotides included in the oligonucleotide set on each of the plurality of nucleic acid sequences.

In order to confirm whether the sequences of the oligonucleotide set are matched or mismatched to each of the plurality of nucleic acid sequences, various methods known in the art can be employed. According to the present invention, in order to confirm the presence or absence of a mismatch, the basic local alignment search tool (BLAST) algorithm usable from the National Center for Biotechnology Information (NCBI) is applied to a program according to the present invention, so that the parameters (e.g., identity, word size, E-value, and the like) used in the BLAST algorithm are modified and used. BLAST is available at http://www.ncbi.nlm.nih.gov/BLAST/. A comparison of sequence similarity using this program may be found at http://www.ncbi.nlm.nh.gov/BLAST/blast_help.html.

In confirming a mismatch between each of the oligonucleotides included in the oligonucleotide set and the nucleic acid sequence, a mismatch between the entire length of the oligonucleotide and a region of the nucleic acid sequence corresponding thereto may be confirmed. As for a tagging probe comprising a tagging portion and a targeting portion, a mismatch between the targeting portion of the tagging probe and a region of the nucleic acid sequence corresponding thereto may be confirmed. As for a DPO primer containing 5′-high Tm specificity portion-separation portion −3′-low Tm specificity portion, a mismatch between the 5′-high Tm specificity portion and a region of the nucleic acid sequence corresponding thereto may be confirmed while a mismatch between the separation portion and a region of the nucleic acid sequence corresponding thereto is not confirmed (that is, considered to be matched), and a mismatch between the 3′-low Tm specificity portion and a region of the nucleic acid sequence corresponding thereto may be confirmed. As for a universal base primer (UBP) or an inosine primer (IPm), a mismatch between a position having a universal base (specifically, inosine) nucleotide in the primer and a nucleotide sequence corresponding thereto is not confirmed (i.e., considered to be matched), and a mismatch between a region of the primer having a natural base and a nucleic acid sequence corresponding thereto is confirmed.

It is confirmed whether the sequences of the oligonucleotide set are mismatched to the plurality of nucleic acid sequences contained in the nucleotide database by using BLAST applied to the program of the present invention, and thus match or mismatch information and position information of each of the oligonucleotides included in the oligonucleotide set for each of the plurality of nucleic acid sequences can be provided.

Since the oligonucleotides included in the oligonucleotide set may have different match or mismatch degrees with respect to the nucleic acid sequences, each of the oligonucleotides has match or mismatch information and position information thereof. The match or mismatch information indicates the number of matches or mismatches and/or a mismatch pattern of each of the oligonucleotides included in the oligonucleotide set for each of the plurality of nucleic acid sequences.

As used herein, the “mismatch pattern” is determined on the basis of information on a position and a base with respect to a mismatch occurring between an oligonucleotide and a nucleic acid sequence.

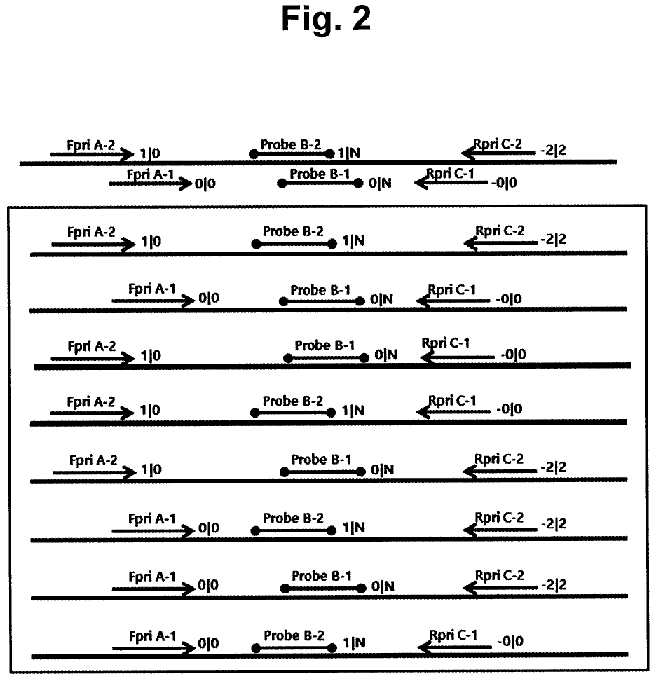

As for FIG. 2, by confirming whether an oligonucleotide set including forward primers (Fpri A-1 and Fpri A-2), probes (Probe B-1 and Probe B-2), and reverse primers (Rpri C-1 and Rpri C-2) is mismatched to a nucleic acid sequence, match or mismatch information (match or mismatch type) and position information (position on the nucleic acid sequence) of each of the oligonucleotides included in the oligonucleotide set can be identified.

As shown in the upper part of FIG. 2, as for match information of the forward primer Fpri A-1, the probe Probe B-1, and the reverse primer Rpri C-1, the number of mismatches is 0, and in other words, the match types thereof are expressed as 0|0, 0|N, and −0|0, respectively. As for mismatch information of the forward primer Fpri A-2, the probe Probe B-2, and the reverse primer Rpri C-2, the numbers of mismatches are 1, 1, and 4, respectively, and in other words, the mismatch types thereof are expressed as 1|0, 1|N, and −2|2, respectively.

The position information of the forward primers (Fpri A-1 and Fpri A-2), probes (Probe B-1 and Probe B-2), and reverse primers (Rpri C-1 and Rpri C-2) is expressed as positions on a nucleic acid sequence, and specifically, expressed as a start point and an end point on a nucleic acid as shown in Table 5 below.

In the match or mismatch type 0|N or −2|2 as match or mismatch information, the bar represents a separation portion of the above-described DPO primer, the portion in the front of the bar represents a 5′-high Tm specificity portion, and the portion in the rear of the bar represents a 3′-low Tm specificity portion, and the numbers of mismatches may be put in the front and rear portions on the basis of the bar. If there are no mismatches, that is, all matches, 0 is put, and if there are two mismatches, 2 is put in the corresponding portion. The meaning that N is marked in the rear portion of the bar is that an oligonucleotide confirmed for a mismatch or match has a structure of a conventional primer or probe which does not have the structure of the DPO primer. The negative (−) sign in the −0|0 or −2|2 as match or mismatch information indicates that it has been confirmed whether an oligonucleotide sequence as a query sequence and a nucleic acid sequence contained in the nucleotide database have a reverse or reverse complementary mismatch therebetween.

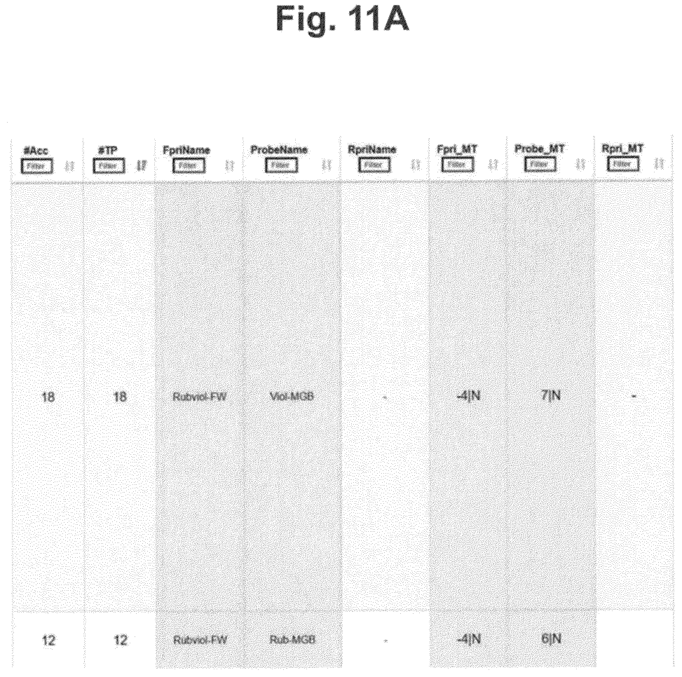

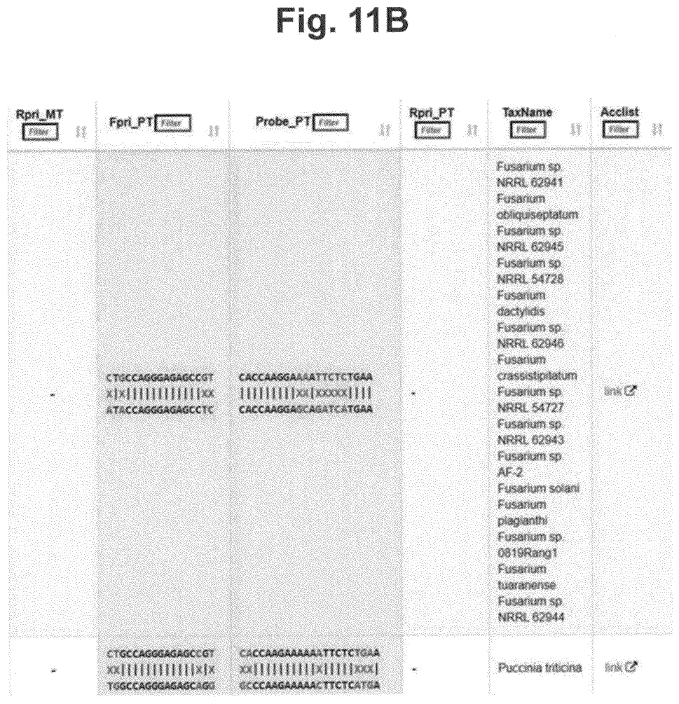

In addition, the mismatch pattern as the match or mismatch information indicates matched and mismatched bases between an oligonucleotide and a nucleic acid sequence corresponding thereto, as shown in FIG. 11B.

For example, as a result of confirming the mismatch or match of a forward primer (Rubviol-FW) with a nucleotide database collected by Trichophton (Taxonomy ID: 5550), it can be seen that the forward primer (Rubviol-FW) has match information of 0|N in which the number of mismatches between positions 182 and 198 of a nucleic acid sequence of accession ID of Z97993.1 is 0 (see Table 5).

Since the nucleic acid sequence confirmed for a mismatch or match is a genomic sequence containing a nucleic acid sequence of a particular target molecule for which the oligonucleotide set is designed, the oligonucleotides included in the oligonucleotide set and confirmed for a mismatch or match may have match information of 01N or 010 in a particular region for which the oligonucleotide set is designed and mismatch information according to the degree of mismatches in another region, and thus even one oligonucleotide may have a plurality of different match or mismatch information and position information for a nucleic acid sequence that has been confirmed for a mismatch or match.

In confirming a mismatch between an oligonucleotide included in the oligonucleotide set and a nucleic acid sequence, the direction of the nucleic acid may be in the order of 5′ to 3′ or 3′ to 5′ when the direction of the oligonucleotide is in the order of 5′ to 3′.

### Step (d): Confirming Whether Probe-Hybridized Amplicons are Generated by Oligonucleotide Set (140)

In the method of the present invention, it is confirmed whether probe-hybridized amplicons by the oligonucleotide set are generated for each of the plurality of nucleic acids. The primer pair includes a forward primer and a reverse primer. The probe-hybridized amplicon is a product amplified by the forward primer and/or the reverse primer, and represents an amplicon that is detected by hybridization of a probe included in the oligonucleotide set. At least one probe-hybridized amplicon is formed by a combination of oligonucleotides according to match or mismatch information and position information of each of the oligonucleotides included in the oligonucleotide set.

One of the main features of the present invention is to analyze the coverage in the unit of a combination of oligonucleotides generating probe-hybridized amplicons, in analyzing the coverage of an oligonucleotide set for a plurality of nucleic acid sequences. This is because the amplicon has a significant meaning when the probe is hybridized to an amplicon to be capable of being amplified by forward and/or reverse primers and such an amplicon is detected.

The probe-hybridized amplicon in the present invention is a product amplified by the forward primer and/or reverse primer, and represents an amplicon that is hybridized with the probe included in the oligonucleotide set and detected. Therefore, a probe needs to be necessarily considered in the amplicon of the present invention.

According to an embodiment of the present invention, the probe-hybridized amplicons in step (d) are generated in the order of the forward primer and the probe, the probe and the reverse primer or the forward primer, the probe and the reverse primer.

Since each of the oligonucleotides included in the oligonucleotide set confirmed for a mismatch or match in step (c) has position information on the nucleic acid sequence, the order of a forward and/or a reverse primer and a probe may be considered as a generation criterion of probe-hybridized amplicons. For example, as for the generation of probe-hybridized amplicons in consideration of the position information of each oligonucleotide, when the forward primer is positioned after the probe or the reverse primer is positioned before the probe, probe-hybridized amplicons are not generated according to the above generation criterion.

In another embodiment, when the oligonucleotide set in step (a) further includes at least one oligonucleotide selected from oligonucleotides consisting of at least one forward primer, at least one probe, and at least one reverse primer, the order of oligonucleotides may be considered as a generation criterion of probe-hybridized amplicons. For example, when there are two forward primers, one probe, or one reverse primer, and each of the oligonucleotides has position information of the order of forward primer 1-probe-forward primer 2-reverse primer, two amplicons may be generated not considering the order as a generation criterion. However, in the present invention, the probe needs to be necessarily included and the predetermined order needs to be considered, and thus probe-hybridized amplicons generated by the order of forward primer 1-probe-reverse primer are probe-hybridized amplicons satisfying the generation criterion of the present invention.

Since each of the oligonucleotides included in the oligonucleotide set may have a plurality of match or mismatch information and position information, at least one probe-hybridized amplicon may be generated for each of nucleic acid sequences by a combination of oligonucleotides according to match or mismatch information and position information of each of the oligonucleotides.

For example, an oligonucleotide set including a forward primer, a probe, and a reverse primer may have a combination of oligonucleotides, which generates probe-hybridized amplicons according to each match or mismatch information on three regions (position information) of a nucleic acid sequence: Region 1 (1∥, 0|N, −2∥), region 2 (0|0, 0|N, −0|0), and Region 3 (5|0, 3|N, 4|2).

According to an embodiment of the present invention, the probe-hybridized amplicons in step (d) are generated or selected to satisfy at least one (specifically two) of the following criteria:

(i) a probe-hybridized amplicon length being less than a predetermined value, wherein this length indicates a length from the nucleotide at the 5′-end of a forward and/or reverse primer to the nucleotide at the 3′-end of an amplicon amplified by the forward and/or reverse primer; and

(ii) the number of mismatches of each of oligonucleotides included in a combination of oligonucleotides being less than a predetermined value.

Specifically, the probe-hybridized amplicons may be generated based on a predetermined order of a forward primer and/or a reverse primer including a probe, and may also be generated or selected by using criteria (i) and (ii) as generation or selection criteria. Therefore, criteria (i) to (ii) are both creation criteria and selection criteria. For example, when criteria (i) and (ii) are generation criteria, the probe-hybridized amplicons according to the present invention may be generated to satisfy at least one of criteria (i) and (ii), in addition to the criterion regarding the predetermined order, and when criteria (i) and (ii) are selection criteria, the probe-hybridized amplicons according to the present invention may be generated to satisfy the criterion regarding the predetermined order and then selected to satisfy at least one of criteria (i) and (ii). Alternatively, one of criteria (i) and (ii) may be a generation criterion, and the other criterion may be a selection criterion. For example, criterion (i) of criteria (i) and (ii) may be a generation criterion, and criterion (ii) may be a selection criterion.

As for the criterion regarding the probe-hybridized amplicon length being less than a predetermined value, which corresponds to criterion (i) of the generation or selection criteria, the length indicates a length from the nucleotide at the 5′-end of a forward and/or reverse primer to the nucleotide at the 3′-end of an amplicon amplified by the forward and/or reverse primer.

The expression “amplicons amplified by the forward and/or reverse primer” was used while expressing the probe-hybridization amplicon length, but when probe-hybridized amplicons are generated in a predetermined order of a forward and/or reverse primer including a probe, this expression encompasses: 1) the length from the nucleotide at the 5′-end of the forward primer to the nucleotide at the 5′-end of the reverse primer, or the nucleotide at the 5′-end or 3′-end of a nucleic acid sequence for the forward primer; 2) the length from the nucleotide at the 5′-end of the reverse primer to the nucleotide at the 5′-end of the forward primer, or the nucleotide at the 5′-end or 3′-end of a nucleic acid sequence for the reverse primer.

The probe-hybridized amplicon length in criterion (i) may be selected in consideration of the performance of DNA polymerase used in PCR or the like. Specifically, the length of less than a predetermined value may be selected in the range of 700 bp to 2000 bp, and may be, for example, less than 700 bp, less than 800 bp, less than 900 bp, less than 1000 bp, less than 1100 bp, less than 1200 bp, less than 1300 bp, less than 1400 bp, less than 1500 bp, less than 1600 bp, less than 1700 bp, less than 1800 bp, less than 1900 bp, or less than 2000 bp.

The terms “nucleotide”, base, bp or mer used while mentioning the length herein may be used interchangeably.

As for the lower limit of the probe-hybridized amplicon length in criterion (i), the lower limit is not important when compared with the upper limit as long as the forward and/or reverse primers and the probe generate probe-hybridized amplicons. Specifically, the lower limit of the probe-hybridized amplicon in criterion (i) may be selected in the range of 100 bp to 600 bp, and may be, for example, 100 bp or more, 150 bp or more, 200 bp or more, 250 bp or more, 300 bp or more, 350 bp or more, 400 bp or more, 450 bp or more, 500 bp or more, 550 bp or more, or 600 bp or more.

As for the criterion regarding the number of mismatches of each of oligonucleotides included in a combination of oligonucleotides being less than a predetermined value, which corresponds to criterion (ii) of the generation or selection criteria, the predetermined value with respect to the number of mismatches may be selected in the range that can cover a designed region of the oligonucleotide set for a target nucleic acid sequence. The predetermined value may be selected in the range of 2 to 15, and may be specifically 2, 3, 4, 5, 6, 7, 8, 9, 10, 11, 12, 13, 14, or 15. When the number of mismatches is expressed as, for example, 312, the predetermined value in the number of mismatches may be the sum of the numbers of mismatches in the front part and the rear part on the basis of the bar, or may represent the number of mismatches in the front or rear part.

The lower limit of the predetermined value in the number of mismatches is preferable as the number of mismatches is smaller.

A plurality of probe-hybridized amplicons may be formed according to the combination of oligonucleotides included in the oligonucleotide set of step (a), and in this case, an additional selection step may be required.

It can be seen that the oligonucleotide set in FIG. 2 includes two forward primers (Fpri A-1 and Fpri A-2), two probes (Probe B-1 and Probe B-2), and two reverse primers (Rpri C-1 and Rpri C-2), and has match or mismatch information for other regions (having different position information) for a nucleic acid sequence. The lower part of FIG. 2 shows that eight probe-hybridized amplicons are generated by combining oligonucleotides having the information. In such a case, a main probe-hybridized amplicon needs to be selected.

According to still another embodiment of the present invention, the method further includes, after step (d), d-1) selecting, as a main probe-hybridized amplicon, a probe-hybridized amplicon satisfying at least one of selection criteria considering the following priorities from at least one formed probe-hybridized amplicon:

(i) a ratio of the sum of the number of mismatches and the number of partial nucleotides in oligonucleotides included in a combination of oligonucleotides relative to the number of the oligonucleotides included in the combination of oligonucleotides, wherein the oligonucleotides included in the combination of oligonucleotides include a probe and a forward primer and/or a reverse primer, and the lower the ratio, the higher the priority;

(ii) the number of mismatches in a probe included in a combination of oligonucleotides, wherein the smaller the number, the higher the priority;

(iii) a ratio of the number of mismatches in a region from the 3′-end of a primer to a nucleotide spaced apart from the 3′-end of the primer by a predetermined length relative to the number of primers included in a combination of oligonucleotides, wherein the lower the ratio, the higher the priority; and

(iv) the number of mismatches in a primer included in a combination of oligonucleotides, wherein the smaller the number, the higher the priority.

According to the present embodiment, from the at least one formed probe-hybridized amplicon, a probe-hybridized amplicon satisfying the selection criteria considering at least one (specifically, a selection criterion considering priority (i)), specifically at least two, more specifically at least three, and most specifically four of the priorities is selected as a main probe-hybridized amplicon.

According to an embodiment of the present invention, the selection criteria considering at least two priorities have a difference in criticality, and the method of the present invention further includes a step of selecting, as a main probe-hybridized amplicon, a probe-hybridized amplicon satisfying the selection criteria considering at least two priorities according to criticality.

There may be largely two manners with respect to a method of selecting a more suitable main probe-hybridized amplicon:

According to the first manner, the selection criteria considering at least two priorities have a difference in criticality, and a main probe-hybridized amplicon satisfying the selection criterion considering the highest criticality (e.g., a selection criterion considering priority (i)) may be selected.

If a plurality of probe-hybridized amplicons satisfy the selection criterion considering the highest priority, a probe-hybridized amplicon satisfying the selection criterion considering the next ranked priority is selected as a main probe-hybridized amplicon.

For example, when the criticality in the selection criteria considering the priorities is in the order of priorities (i), (ii), (iii), and (iv) and three probe-hybridized amplicons satisfy the selection criterion considering priority (i), it is confirmed whether these three probe-hybridized amplicons satisfy the selection criterion considering priority (ii). If the selection criterion considering priority (ii) is satisfied, a probe-hybridized amplicon satisfying the selection criterion considering priority (iii) is selected as a main probe-hybridized amplicon.

According to the second manner, when different weights are assigned to the selection criteria considering priorities and scores are assigned to values (or value ranges) in each of the selection criteria considering priorities, the total score of each of the probe-hybridized amplicons can be obtained. Considering the calculated total score, a main probe-hybridized amplicon may be selected.

As for the selection criterion considering priority (i) among the selection criteria, a probe-hybridized amplicon in which the ratio of the sum of the number of mismatches and the number of partial nucleotides in oligonucleotides included in a combination of oligonucleotides generating probe-hybridized amplicons relative to the number of oligonucleotides is lowest, is selected as a main probe-hybridizing amplicon.

The sum of the number of mismatches and the number of partial nucleotides in the oligonucleotides indicates the sum of the number of mismatches and the number of partial nucleotides in each of the oligonucleotides.

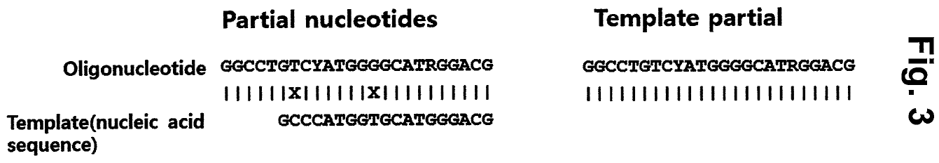

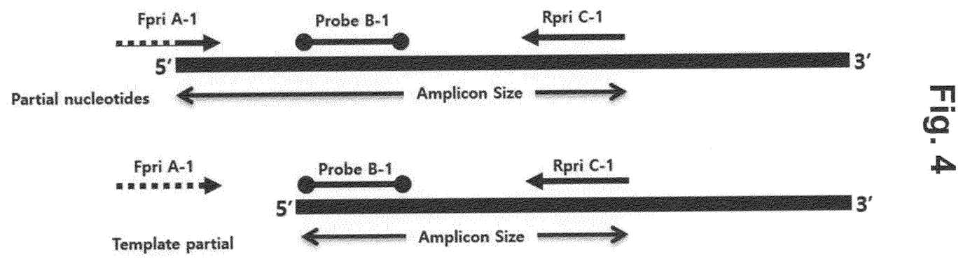

As shown in FIGS. 3 and 4, the partial nucleotides represent nucleotides of a nucleic acid sequence without a part when a sequence of a nucleic acid sequence (template) with respect to the oligonucleotide sequence is partially absent. In FIG. 3, the number of partial nucleotides is 5, which may be expressed as 5′(5). The five partial nucleotides in the 3′-part of the nucleic acid sequence are expressed as 3′(5). In FIG. 3, the number of mismatches in the oligonucleotide for the nucleic acid sequence having partial nucleotides is expressed as 2|N in consideration of the number of mismatches in the part excluding the partial nucleotides. The template partial indicates the absence of a sequence in a region of the nucleic acid sequence corresponding to the oligonucleotide sequence, as can be shown in FIGS. 3 and 4, and this is expressed as “−”.

As for the selection criterion considering priority (ii) among the selection criteria, the smaller the number of mismatches of a probe included in a combination of oligonucleotides, the higher the priority.

Next, as for the selection criterion considering priority (iii), the lower the ratio of the number of mismatches in a region from the 3′-end of the primer to a nucleotide spaced apart from the 3′-end of the primer by a predetermined length relative to the number of primers included in a combination of oligonucleotides, the higher the priority. The predetermined length, mentioned when a nucleotide spaced apart from the 3′-end of the primer by a predetermined length is expressed, may be selected in the range of 6 to 15 nucleotides.

In a primer having a structure of a DPO primer in which the number of mismatches (mismatch type) in an oligonucleotide is expressed as, for example, 0∥, the region from the 3′-end of the primer to a nucleotide spaced apart therefrom by a predetermined length indicates a 3′-low Tm specificity portion, and corresponds to the rear part on the basis of the bar.

As for the selection criterion considering priority (iv) among the selection criteria, the smaller the number of mismatches of a primer included in a combination of oligonucleotides, the higher the priority. In a case where there are a plurality of primers, the number of mismatches in the primer indicates the sum of the numbers of mismatches in the respective primers. When the primer has a structure of a DPO primer, the higher the priority of the mismatch type of the primer, the higher the priority. The priority of mismatch type of the primer decreases in the order of, for example, 0|0, 1|0, 0|1, 1|1, 2|0, 2|1 and 0|2.

For example, in an oligonucleotide set including a forward primer, a probe, and a reverse primer, combinations of oligonucleotides generating probe-hybridized amplicons are as follows: Combination 1 (0|0, 2|N, 0|1), Combination 2 (5′(2) 0|0, 1|N, 0|1), Combination 3 (−, 2|N, 1|1), Combination 4 (−, 2|N, 0|0), Combination 5 (1|0, 1|N, 0|1), Combination 6 (−, 1|N, 0|1), and Combination 7 (0|0, 1|N, 1|1). If the selection criteria considering the priorities has the criticality in the order of priorities (i), (ii), (iii) and (iv), a main probe-hybridized amplicon may be selected as follows:

The ratio according to the selection criterion of (i) among the selection criteria is 1 for Combination 1, 1.3 for Combination 2, 2 for Combination 3, 1 for Combination 4, 1 for Combination 5, 1 for Combination 6, and 1 for Combination 7. The probe-hybridized amplicons generated by Combinations 1, 4, 5, 6, and 7 satisfy the selection criterion of (i). For example, the ratio according to the selection criterion of (i) in Combination 2 is 4/3 or 1.3 since the number of oligonucleotides constituting the combination is 3 (a forward primer, a probe, and a reverse primer) and the sum of the numbers of mismatches and partial nucleotides in the respective oligonucleotides is 4.

Considering the number of mismatches of a probe as selection criterion (ii) for Combinations 1, 4, 5, 6, and 7, the number is 2 for Combination 1, 2 for Combination 4, and 1 for Combinations 5 to 7, and thus the probe-hybridized amplicons generated by Combinations 5 to 7 are selected.

Considering the ratio of the number of mismatches in a predetermined region from the 3′-end of a primer as selection criterion (iii) for Combinations 5 to 7, the ratio is ½ or 0.5 for Combination 5, 1/1 or 1 for Combination 6, and ½ or 0.5 for Combination 7, and thus the probe-hybridized amplicons generated by Combinations 5 and 7 are selected.

Considering the number of mismatches in a primer as selection criterion (iv) for Combinations 5 and 7, the primers have a structure of a DPO primer, and thus when the priority of the mismatch type of a primer is considered, the priority of the mismatch type (0|0) of the forward primer of Combination 7 is higher than the priority of the mismatch type (1|0) of Combination 5. Therefore, a probe-hybridized amplicon generated by Combination 7 is finally selected as a main probe-hybridized amplicon.

According to an embodiment of the present invention, the method further includes a step of selecting as a candidate probe-hybridized amplicon a probe-hybridized amplicon generated by a combination of oligonucleotides, which has the sum of the numbers of mismatches within a predetermined number to the sum of the numbers of mismatches of oligonucleotides included in a combination of the oligonucleotides generating the main probe-hybridized amplicon. The sum of the numbers of mismatches within a predetermined number represents the sum obtained by adding a predetermined number or less to the sum of the numbers of mismatches of oligonucleotides included in a combination of the oligonucleotides generating the main probe-hybridized amplicon. Specifically, the predetermined number may be selected from 2 to 8, and may be for example 2, 3, 4, 5, 6, 7, or 8.

When the present embodiment is applied to Table 5, a probe-hybridized amplicon generated by the forward primer (Rubviol-FW), probe (Rub-MGB), and reverse primer (Rubviol-REV) is a main probe-hybridized amplicon, and a probe-hybridized amplicon generated by the forward primer (Rubviol-FW), probe (Viol-MGB), and reverse primer (Rubviol-REV) is a candidate probe-hybridized amplicon. The probe (Viol-MGB) is a candidate oligonucleotide.

One of the features of the present invention is to provide information on mismatch patterns in the unit of a combination of oligonucleotides by grouping the mismatch patterns of each of the oligonucleotides included in a combination of oligonucleotides generating probe-hybridization amplicons.

According to an embodiment of the present invention, the method further includes the following step, after step (d): d-2) grouping, according to sequence identity, sequences having a mismatch pattern for each of types of oligonucleotides included in the combination of the oligonucleotides, wherein the grouped sequences having a mismatch pattern has a mismatch pattern having the same mismatch position between the oligonucleotide and the nucleic acid sequence in oligonucleotides of the same type having the mismatch pattern and has a mismatch pattern having the same base between oligonucleotide sequences and between nucleic acid sequences at the mismatch position; and the oligonucleotide type indicates a type of oligonucleotides as a forward primer, a probe, and a reverse primer; and d-3) providing information on the mismatch pattern for each combination of oligonucleotides having the same mismatch pattern(or type) and generating probe-hybridized amplicons by combining oligonucleotides having the grouped sequences having a mismatch pattern, wherein the information on the mismatch pattern indicates oligonucleotide sequences and nucleic acid sequences having the mismatch pattern, the number of nucleic acid sequences having the mismatch pattern, or a list of identifiers.

As for the grouping in step d-2) according to the embodiment, the grouping is not performed when the match or mismatch type is 010 or OWN for each of types of oligonucleotides, but the grouping is performed on oligonucleotides having a mismatch. Specifically, sequences having the same mismatch pattern are grouped for each type of oligonucleotides having mismatch patterns. The grouped sequences having a mismatch pattern has a mismatch pattern having the same mismatch position between the oligonucleotide and the nucleic acid sequence in oligonucleotides of the same type having the mismatch pattern and has a mismatch pattern having the same base between oligonucleotide sequences and between nucleic acid sequences at the mismatch position.

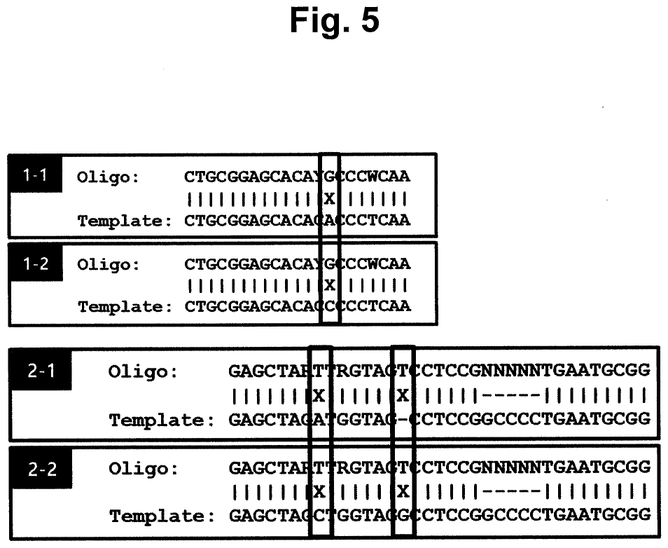

As shown in FIG. 5, although the mismatch position between the oligonucleotide and the nucleic acid sequence is the same in the same type of oligonucleotides having mismatch patterns, the mismatch patterns are not the same if different bases are present or if a gap is present at the mismatch position between oligonucleotide sequences and between nucleic acid sequences.

An another embodiment, as long as the mismatch position between an oligonucleotide and a nucleic acid sequence is the same between the same type of oligonucleotides having mismatch patterns and the bases at the mismatch position are same between oligonucleotide sequences and between nucleic acid sequences, the mismatch patterns are grouped as having the same mismatch pattern although there is a difference in view of the presence or absence of partial nucleotides in a region of the nucleic acid sequence matched between the oligonucleotide and the nucleic acid sequence in the same type of oligonucleotides having mismatch patterns.

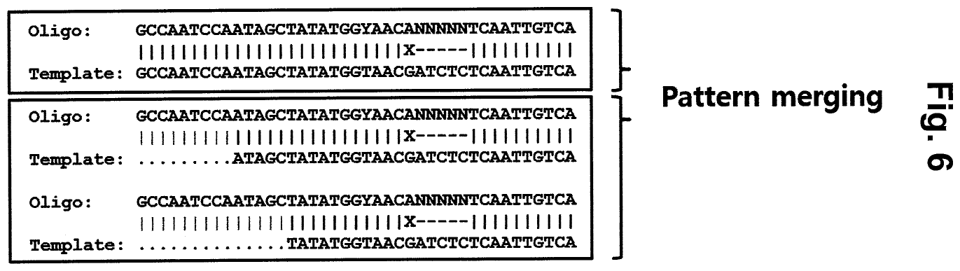

As shown in FIG. 6, when a difference in mismatch pattern is only the presence or absence of partial nucleotides in a region of the nucleic acid sequence matched between the oligonucleotide and the nucleic acid sequence, the part with partial nucleotides is regarded as being a match, and is grouped with the same mismatch pattern.

In the present invention, the coverage of the oligonucleotide set is analyzed in the unit of a probe-hybridized amplicon, and thus information on mismatch patterns is also provided for each combination of oligonucleotides generating probe-hybridized amplicons.

FIGS. 11A and 11B show mismatch patterns of probe-hybridized amplicons of oligonucleotide set 1 with respect to a nucleotide database including nucleic acid sequences of Trichophyton. As can be seen from FIGS. 11A and 11B, information on mismatch patterns, including oligonucleotide sequences and nucleic acid sequences having the mismatch patterns, the number of nucleic acid sequences having the mismatch patterns, and a list of identifiers, can be identified.

Step (e): Providing Nucleic Acid Sequences with Generation of Probe-Hybridized Amplicons by Oligonucleotide Set and/or Nucleic Acid Sequences without Generation of Probe-Hybridized Amplicons by Oligonucleotide Set (150)

Last, the method of the present invention provides nucleic acid sequences with the generation of probe-hybridized amplicons by the oligonucleotide set and/or nucleic acid sequences without the generation of probe-hybridized amplicons by the oligonucleotide set. The nucleic acid sequences with the generation of probe-hybridized amplicons are covered by the oligonucleotide set, and the nucleic acid sequences without the generation of probe-hybridized amplicons are not covered by the oligonucleotide set.

As used herein, the term “coverage” refers to providing information on nucleic acid sequences hybridized or covered by a combination of forward and/or reverse primers, and probes included in an oligonucleotide set.

According to an embodiment of the present invention, the nucleic acid sequences in step (e) contain information on nucleic acid sequences selected from the group consisting of the number of nucleic acid sequences, accession numbers (Accession Nos.) of the nucleic acid sequences, taxonomy names to which the nucleic acid sequences belong, taxonomy IDs assigned to the taxonomy names, ratios of nucleic acid sequences covered by combinations of the oligonucleotides relative to the total nucleic acid sequences, and mismatch patterns of oligonucleotides included in the combination of the oligonucleotides.

The nucleic acid sequences with the generation of probe-hybridized amplicons by the oligonucleotide set in step (e) may be classified based on predetermined criteria.

According to an embodiment of the present invention, the nucleic acid sequences with the generation of probe-hybridized amplicons by the oligonucleotide set in step (e) include: nucleic acid sequences satisfying the following criteria (i) and (ii) (probe-hybridized amplicon match nucleic acid sequences); and nucleic acid sequences satisfying the following criteria (iii) and (iv) (probe-hybridized amplicon mismatch nucleic acid sequences):

(i) a predetermined probe-hybridized amplicon length range;

(ii) the number of mismatches for nucleic acid sequences in all of the oligonucleotides included in a combination of the oligonucleotides being 0 (zero);

(iii) exceeding the length range of the above (i); and

(iv) the number of mismatches in at least one oligonucleotide of the oligonucleotides included in a combination of the oligonucleotides being less than a predetermined value.

The nucleic acid sequences with the generation of probe-hybridized amplicons are largely divided into probe-hybridized amplicon match nucleic acid sequences and probe-hybridized amplicon mismatch nucleic acid sequences according to the criteria regarding the predetermined probe-hybridized amplicon length range and the presence or absence of mismatches.

First, the probe-hybridized amplicon match nucleic acid sequences satisfy the criteria regarding (i) a predetermined probe-hybridized amplicon length range and (ii) the number of mismatches for nucleic acid sequences in all the oligonucleotides included in a combination of the oligonucleotides being 0 (zero).

According to an embodiment of the present invention, the predetermined probe-hybridized amplicon length range (i) is determined by a user or determined by a probe-hybridized amplicon length of high frequency among the lengths of the probe-hybridized amplicons generated by a combination of oligonucleotides having no mismatches for a plurality of nucleic acid sequences.

The probe-hybridized amplicon length range may be determined by a user. For example, when the probe-hybridized amplicon length is determined to be 200 bp and the length range is determined to be 200 bp±50% by a user, the probe-hybridized amplicon length is 100 bp≤probe-hybridized amplicon length≤300 bp. Alternatively, the probe-hybridized amplicon length range is determined by a probe-hybridized amplicon length of high frequency among the lengths of the probe-hybridized amplicons generated by a combination of oligonucleotides having no mismatches for a plurality of nucleic acid sequences. More specifically, the probe-hybridized amplicon length range is determined by the probe-hybridized amplicon length of highest frequency, and under the same frequency, the length range is determined by an average length.

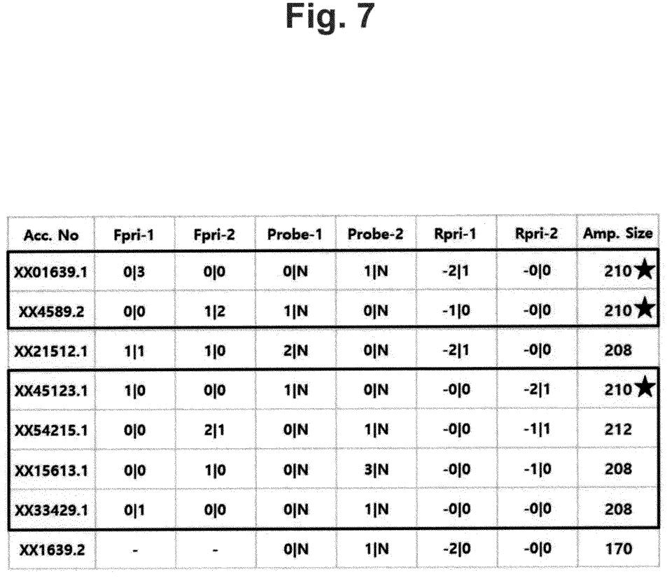

Referring to FIG. 7, probe-hybridized amplicon lengths for a plurality of nucleic acid sequences, by combinations of oligonucleotides generating probe-hybridized amplicons and all having match types through the combination of forward primers Fpri-1 and Fpri-2, probes Probe-1 and Probe-2, and reverse primers Rpri-1 and Rpri-2, are calculated, and among the calculated lengths, the probe-hybridized amplicon length of highest frequency is selected. In FIG. 7, the probe-hybridized amplicon length of highest frequency by all oligonucleotides combined in match types is 210 bp, and a predetermined length range, 210 bp±50%, that is, 105 bp≤probe-hybridized amplicon length≤315 bp is then determined therefrom. The predetermined probe-hybridized amplicon length range is specifically the probe-hybridized amplicon length ±20%, ±30%, ±40%, ±50%, ±60%, ±70%, or ±80%. Specifically, the probe-hybridized amplicon length (bp) is selected from 100 bp to 600 bp.

As for the criterion (ii) regarding the number of mismatches for nucleic acid sequences in all of the oligonucleotides included in a combination of the oligonucleotides being 0 (zero), this means that all of the oligonucleotides included in the combination of oligonucleotides generating probe-hybridized amplicons need to have match types.

For example, when the lengths of the probe-hybridized amplicons generated by a combination of oligonucleotides are within a predetermined length range and all of the oligonucleotides included in the combination have match types, a plurality of nucleic acid sequences with respect to the combination of the oligonucleotides are classified as probe-hybridized amplicon match nucleic acid sequences. In addition, the nucleic acid sequences with the generation of probe-hybridized amplicons are covered by the oligonucleotide set.

As used herein, the term “cover” means that an oligonucleotide set (a primer pair and a probe) is sufficiently complementary to be selectively hybridized with a target nucleic acid sequence under designated annealing conditions or stringent conditions, and the term encompasses the terms “substantially complementary” and “perfectly complementary”. Specifically, the term “cover” herein means being perfectly complementary.

As used herein, the term “hybridization” means that a double-stranded nucleic acid is formed from a complementary single-stranded nucleic acid. An oligonucleotide set to be hybridized with a target nucleic acid sequence includes not only a sequence perfectly complementary to the target nucleic acid sequence but also a sequence that is sufficient to be specifically hybridized with the target nucleic acid sequence under particular stringent conditions. For example, an oligonucleotide set may include one or more non-complementary nucleotides (i.e., mismatches) to a target nucleic acid sequence as long as its specificity is not impaired. Therefore, the oligonucleotide set in the present invention may include partially and completely complementary sequences to a target nucleic acid sequence, and particularly, include a perfectly complementary sequence (or a matching sequence).

The probe-hybridized amplicon match nucleic acid sequences contain information on nucleic acids selected from the group consisting of the number of nucleic acid sequences, accession numbers (Accession Nos.) of the nucleic acid sequences, taxonomy names to which the nucleic acid sequences belong, and taxonomy IDs assigned to the taxonomy names. For example, a user can provide a coverage by providing taxonomy names of nucleic acid sequences covered by an oligonucleotide set through the information of probe-hybridized amplicon match nucleic acid sequences.

In an embodiment, when the oligonucleotide set in step (a) further includes at least one oligonucleotide selected from the oligonucleotides consisting of at least one forward primer, at least one probe, and at least one reverse primer and a probe-hybridized amplicon satisfying selection criteria considering priorities is selected as a main probe-hybridized amplicon from the at least one formed probe-hybridized amplicon, criterion (i) for the probe-hybridized amplicon match nucleic acid sequences is used to confirm whether the length of the main probe-hybridized amplicon falls within the predetermined length range, and criterion (ii) is applied to a combination of oligonucleotides generating the main probe-hybridized amplicon.

Meanwhile, nucleic acid sequences having the generation of probe-hybridized amplicons but satisfying the following criteria (iii) and (iv) are classified as probe-hybridized amplicon mismatch nucleic acid sequences: (iii) exceeding the length range of the above (i); and (iv) the number of mismatches in at least one oligonucleotide of the oligonucleotides included in a combination of the oligonucleotides being less than a predetermined value.

Although all the oligonucleotides included in a combination of oligonucleotides generating probe-hybridized amplicons have match types, nucleic acid sequences satisfying the criterion regarding exceeding the length range of the above (i) are provided as probe-hybridized amplicon mismatch nucleic acid sequences. Although the length of the probe-hybridized amplicons satisfies the length range of the above (i), nucleic acid sequences satisfying criterion (iv) regarding the number of mismatches in at least one oligonucleotide of the oligonucleotides included in a combination of the oligonucleotides being less than a predetermined value are provided as probe-hybridized amplicon mismatch nucleic acid sequences.

The predetermined value in the number of mismatches may be selected in the range of 1 to 8, and may be specifically 1, 2, 3, 4, 5, 6, 7, or 8.

The probe-hybridized amplicon mismatch nucleic acid sequences contain information on nucleic acids selected from the group consisting of the number of nucleic acid sequences, accession numbers (Accession Nos.) of the nucleic acid sequences, taxonomy names to which the nucleic acid sequences belong, taxonomy IDs assigned to the taxonomy names, and mismatch patterns of oligonucleotides included in combinations of the oligonucleotides.

For example, when mismatch patterns of oligonucleotides included in a combination of the oligonucleotides among the information on probe-hybridized amplicon mismatch nucleic acid sequences are provided, a degenerate base and/or a universal base is introduced at a mismatch position of an oligonucleotide having the mismatch pattern to modify the oligonucleotide, thereby being capable of improving the coverage of a combination of oligonucleotides including the modified oligonucleotide.

In an embodiment, when the oligonucleotide set in step (a) further includes at least one oligonucleotide selected from the oligonucleotides consisting of at least one forward primer, at least one probe, and at least one reverse primer and a probe-hybridized amplicon satisfying selection criteria considering priorities is selected as a main probe-hybridized amplicon from the at least one formed probe-hybridized amplicons, criterion (iii) for the probe-hybridized amplicon mismatch nucleic acid sequences is used to confirm whether the length of the main probe-hybridized amplicon exceeds the predetermined length range, and criterion (iv) is applied to a combination of oligonucleotides generating the main probe-hybridized amplicon.

In an embodiment of the present invention, when the plurality of nucleic acid sequences contained in the nucleotide database of step (b) are a plurality of target nucleic acid sequences, step (e) provides nucleic acid sequences with the generation of probe-hybridized amplicons by the oligonucleotide set, thereby providing a plurality of target nucleic acid sequences covered by the oligonucleotide set.

Specifically, when a plurality of nucleic acid sequences contained in the nucleotide database are a plurality of target nucleic acid sequences to be amplified and detected by the oligonucleotide set used in the present invention, the nucleic acid sequences with the generation of probe-hybridized amplicons by the oligonucleotide set are provided as a plurality of target nucleic acid sequences covered by the oligonucleotide set. Through this, the coverage for a plurality of target nucleic acids to be amplified and detected can be provided, and thus the specificity of the oligonucleotide set can be provided.

According to another embodiment of the present invention, the plurality of nucleic acid sequences contained in the nucleotide database of step (b) are a plurality of non-target nucleic acid sequences, and step (e) provides information on nucleic acid sequences without the generation of probe-hybridized amplicons by the oligonucleotide set, thereby providing a plurality of non-target nucleic acid sequences not covered by the oligonucleotide set.

Specifically, when a plurality of nucleic acid sequences contained in the nucleotide database are a plurality of non-target nucleic acid sequences to be not amplified and detected by the oligonucleotide set used in the present invention, the nucleic acid sequences without the generation of probe-hybridized amplicons by the oligonucleotide set are provided as a plurality of target nucleic acid sequences not covered by the oligonucleotide set. Through this, the information on the plurality of non-target nucleic acid sequences to be not amplified and detected may be provided.

In an embodiment, in cases where the sequences of the oligonucleotide set are input in steps (a) and a-1), respectively, steps (b) to (e) as descried above are performed on each of the oligonucleotide sets. Therefore, the description of the above-described steps (b) to (e) on the oligonucleotide set inputted in step (a) is also applied to the oligonucleotide set inputted in step a-1) in the same manner.

Therefore, in step (e), provided are: the nucleic acid sequences with the generation of probe-hybridized amplicons, and/or the nucleic acid sequences without the generation of probe-hybridized amplicons by the oligonucleotide set, of which the sequences are inputted in step (a); and the nucleic acid sequences with the generation of probe-hybridized amplicons, and/or the nucleic acid sequences without the generation of probe-hybridized amplicons by the oligonucleotide set, of which the sequences are inputted in step a-1).

In cases where the method of the present invention further includes, after step (a), step a-1) of inputting sequences of at least one oligonucleotide set, which is different from the oligonucleotide set in step (a), the method may further include f) comparing the coverage for the plurality of nucleic acid sequences by the oligonucleotide set in step a) with the coverage for a plurality of nucleic acid sequences by at least one oligonucleotide set in step a-1).

For example, referring to Table 4, nucleic acid sequences with the generation of probe-hybridized amplicons and nucleic acid sequences without the generation of probe-hybridized amplicons for the respective nucleic acid sequences collected under the taxonomy name Trichophyton (Taxonomy ID: 5550) for oligonucleotides 1 and 2 each are provided, and the coverages of oligonucleotide sets 1 and 2 for each of the nucleic acid sequences can be compared. In Table 4 below, it can be confirmed that as for the nucleic acid sequences set forth as accession numbers, the nucleic acid sequences with the generation of probe-hybridized amplicons by oligonucleotide set 1 are more than the nucleic acids by oligonucleotide set 2, and taxonomy name of each of the nucleic acid sequences can be confirmed.

According to another embodiment of the present invention, the oligonucleotide set in step (a) and the at least one oligonucleotide set in step a-1) are oligonucleotide sets designed at different time points.

Specifically, the oligonucleotide set in step (a) may be an oligonucleotide set designed based on the nucleic acid sequences listed in the public-accessible nucleotide database at time point A, and the at least one oligonucleotide set in step a-1) may be an oligonucleotide set designed based on the nucleic acid sequences listed in the public-accessible nucleotide database at time point B after time point A. For example, in a case where an oligonucleotide set used to amplify and detect a plurality of nucleic acid sequences for a certain target nucleic acid molecule is designed, and then after a period of time, an oligonucleotide set used to amplify and detect a plurality of nucleic acid sequences including nucleic acid sequences additionally listed in the nucleotide database is newly designed, the comparison of the coverage of the previously designed oligonucleotide set and the coverage of the newly designed oligonucleotide set needs to be made at the present time point. In this case, the sequences of the previously designed oligonucleotide set and the newly designed oligonucleotide set are inputted and subjected to steps (a) to (f) as described above, thereby being capable of comparing the coverage therebetween.

According to an embodiment of the present invention, the method further includes the following steps: (f) performing steps (a) to (e) on a nucleotide database provided at a different time point from step (b); and (g) comparing the resultant in step (e) and the resultant in step (e) of step (f).

The present embodiment shows that an oligonucleotide set, which has been analyzed for the coverage for a nucleotide database at time point A, is analyzed for the coverage for a nucleotide database provided at time point B, which is a different time point after time point A, and the coverages of the oligonucleotide set at time points A and B are compared.

The resultant in step (e) indicates the nucleic acid sequences with the generation of probe-hybridized amplicons and/or the nucleic acid sequences without the generation of probe-hybridized amplicons provided in step (e) by inputting the sequences of the oligonucleotide set at time point A in step (a) and performing steps (b) to (e) on the nucleotide database provided at time point A. The resultant in step (e) of step (f) indicates the nucleic acid sequences with the generation of probe-hybridized amplicons and/or the nucleic acid sequences without the generation of probe-hybridized amplicons provided at time point B with respect to the oligonucleotide set at time point A by inputting the sequences of the oligonucleotide set, which are the same as the sequences inputted at time point A in step (a), at time point B, providing a nucleotide database containing newly collected nucleic acid sequences at time point B, and then performing steps (c) to (e).

The present embodiment also shows that an oligonucleotide set for detecting a particular organism is designed at time point A, the coverage of the designed oligonucleotide set is analyzed for a nucleotide database for a particular organism, the coverage of the oligonucleotide set is analyzed for a nucleotide database containing a variant sequence for a particular organism provided at time point B, which is a different time point after time point A, and then the coverages of the oligonucleotide set at time points A and B are compared. For example, an oligonucleotide set for detecting SARS-CoV-2 is designed at time point A, the coverage of the designed oligonucleotide set is analyzed for a nucleotide database for SARS-CoV-2, the coverage of the oligonucleotide set is analyzed for a SARS-CoV-2 variant sequence found at time point B, which is a different time point after time point A, and then the coverages of the oligonucleotide set at time points A and B are compared, and when the oligonucleotide set covers the SARS-CoV-2 variant sequence, the oligonucleotide set can be used to amplify and detect the SARS-CoV-2 variant sequence, but when the oligonucleotide set cannot cover the SARS-CoV-2 variant sequence, the oligonucleotides included in the oligonucleotide set need to be modified or replaced by confirming mismatch information.

In addition, in the case of comparing the coverage between the oligonucleotide set at time A and time B, when the oligonucleotide set at time A covers a certain target nucleic acid sequence, but the oligonucleotide set at time B does not cover the certain target nucleic acid sequence, the oligonucleotide set should be modified or replaced (i.e., improved).

The improvement of such an oligonucleotide set depends on the mismatch tolerance for each type of oligonucleotides. Specifically, in the case of a primer having the structure of a DPO primer, the oligonucleotide set does not need to be improved if it has the following mismatch tolerance. Specifically, the most conserved sequence in a conserved region is located at the 3′-end region of the DPO primer, and the lowest conserved sequence is located at the separation portion. The 5′-high Tm specificity portion and/or the 3′-low Tm specificity portion (specifically, the 5′-high Tm specificity portion) may contain one or more, specifically one to three, more specifically one or two mismatched bases at the target site due to the mismatch tolerance of the DPO primer.

As used herein, the term “conserved region” refers to a fragment of the nucleotide sequence of a gene or amino acid sequence of a protein that is substantially similar among various nucleotide sequences. The term is used interchangeably with “conserved sequence”.

### Storage Medium, Device, and Program

In another aspect of the present invention, there is provided a computer readable storage medium containing instructions to configure a processor to perform a method for providing a coverage of an oligonucleotide set for a plurality of nucleic acid sequences, the method including: (a) inputting sequences of an oligonucleotide set, wherein the oligonucleotide set includes a primer pair and a probe as oligonucleotides; (b) providing a nucleotide database, wherein the nucleotide database contains a plurality of nucleic acid sequences; (c) providing match or mismatch information and position information of each of the oligonucleotides included in the oligonucleotide set for each of the plurality of nucleic acid sequences by confirming whether the sequences of the oligonucleotide set are mismatched to the plurality of nucleic acid sequences contained in the nucleotide database, wherein the match or mismatch information indicates the number of matches or mismatches and/or a mismatch pattern of each of the oligonucleotides included in the oligonucleotide set for each of the plurality of nucleic acid sequences; (d) confirming whether probe-hybridized amplicons are generated by the oligonucleotide set for each of the plurality of nucleic acid sequences, wherein the primer pair includes a forward primer and a reverse primer; the probe-hybridized amplicons are products amplified by the forward primer and/or reverse primer and indicate amplicons detected by hybridization of the probe included in the oligonucleotide set; and at least one of the probe-hybridized amplicons is formed by a combination of the oligonucleotides according to the match or mismatch information and position information of each of the oligonucleotides included in the oligonucleotide set for each of the plurality of nucleic acid sequences; and (e) providing nucleic acid sequences with the generation of probe-hybridized amplicons by the oligonucleotide set and/or nucleic acid sequences without the generation of probe-hybridized amplicons by the oligonucleotide set, wherein the nucleic acid sequences with the generation of probe-hybridized amplicons are covered by the oligonucleotide set and the nucleic acid sequences without the generation of probe-hybridized amplicons are not covered by the oligonucleotide set.

In still another aspect of the present invention, there is provided a computer program to be stored on a computer readable storage medium, to configure a processor to perform a method for providing a coverage of an oligonucleotide set for a plurality of nucleic acid sequences, the method including: (a) inputting sequences of an oligonucleotide set, wherein the oligonucleotide set includes a primer pair and a probe as oligonucleotides; (b) providing a nucleotide database, wherein the nucleotide database contains a plurality of nucleic acid sequences; (c) providing match or mismatch information and position information of each of the oligonucleotides included in the oligonucleotide set for each of the plurality of nucleic acid sequences by confirming whether the sequences of the oligonucleotide set are mismatched to the plurality of nucleic acid sequences contained in the nucleotide database, wherein the match or mismatch information indicates the number of matches or mismatches and/or a mismatch pattern of each of the oligonucleotides included in the oligonucleotide set for each of the plurality of nucleic acid sequences; (d) confirming whether probe-hybridized amplicons are generated by the oligonucleotide set for each of the plurality of nucleic acid sequences, wherein the primer pair includes a forward primer and a reverse primer; the probe-hybridized amplicons are products amplified by the forward primer and/or reverse primer and indicate amplicons detected by hybridization of the probe included in the oligonucleotide set; and at least one of the probe-hybridized amplicons is formed by a combination of the oligonucleotides according to the match or mismatch information and position information of each of the oligonucleotides included in the oligonucleotide set for each of the plurality of nucleic acid sequences; and (e) providing nucleic acid sequences with the generation of probe-hybridized amplicons by the oligonucleotide set and/or nucleic acid sequences without the generation of probe-hybridized amplicons by the oligonucleotide set, wherein the nucleic acid sequences with the generation of probe-hybridized amplicons are covered by the oligonucleotide set and the nucleic acid sequences without the generation of probe-hybridized amplicons are not covered by the oligonucleotide set.

In another aspect of the present invention, there is provided a device for providing a coverage of an oligonucleotide set for a plurality of nucleic acid sequences, comprising (a) a computer processor and (b) a computer readable storage medium of the present invention coupled to the computer processor.

Since the storage medium, the device, and the computer program of the prevent invention are intended to perform the present methods described as above in a computer, the common descriptions among them are omitted in order to avoid undue redundancy leading to the complexity of this specification.

The program instructions are operative, when performed by the processor, to cause the processor to perform the present method described above. The program instructions for performing the method of providing a coverage of an oligonucleotide set for a plurality of nucleic acid sequences may include the following instructions: (i) an instruction to input sequences of an oligonucleotide set; (ii) an instruction to provide a nucleotide database; (iii) an instruction to provide match or mismatch information and position information of each of the oligonucleotides included in the oligonucleotide set for each of the plurality of nucleic acid sequences by confirming whether the sequences of the oligonucleotide set are mismatched to the plurality of nucleic acid sequences contained in the nucleotide database; (iv) an instruction to confirm whether probe-hybridized amplicons are generated by the oligonucleotide set for each of the plurality of nucleic acid sequences; and (v) an instruction to provide (e.g., display on an output device) nucleic acid sequences with the generation of probe-hybridized amplicons by the oligonucleotide set and/or nucleic acid sequences without the generation of probe-hybridized amplicons by the oligonucleotide set.

The method of the present invention is implemented in a processor, and the processor may be a processor in a stand-alone computer, a network attached computer, or a data acquisition device, such as a real-time PCR device.

The types of the computer readable storage medium include various storage media, for example, CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory and web server, but are not limited thereto.

The coverage of an oligonucleotide set for a plurality of nucleic acid sequences may be provided in various manners. For example, the coverage of an oligonucleotide set for a plurality of nucleic acid sequences may be provided to a separate system, such as a desktop computer system, via a network connection (e.g., LAN, VPN, intranet, and internet) or a direct connection (e.g., USB or other direct wired or wireless connection), or may be provided on a portable medium such as a CD, DVD, floppy disk and portable HDD. Similarly, the coverage of an oligonucleotide set for a plurality of nucleic acid sequences may be provided to a server system via a network connection (e.g., LAN, VPN, Internet, intranet and wireless communication network) to a client, such as a notebook or a desktop computer system.

The instructions to configure the processor to perform the present invention may be included in a logic system. The instructions may be downloaded and stored in a memory module (e.g., hard drive or other memory, such as a local or attached RAM or ROM), although the instructions can be provided on any software storage medium (e.g., portable HDD, USB, floppy disk, CD and DVD). A computer code for implementing the present invention may be implemented in a variety of coding languages, such as C, C++, Java, Visual Basic, VBScript, JavaScript, Perl and XML. In addition, a variety of languages and protocols may be used in external and internal storage and transmission of data and commands according to the present invention.

The computer processor may be constructed in such a manner that a single processor can make several performances. Alternatively, the processor unit may be constructed in such a manner that several processors make several performances, respectively.

The features and advantages of the present invention are summarized as follows:

(a) Conventionally, the specificity analysis of an oligonucleotide set (e.g., a primer pair and a probe) used to amplify and detect a plurality of target nucleic acid molecules could not be efficiently performed. Specifically, according to a conventional method of performing specificity analysis in the unit of an oligonucleotide, the specificity is analyzed for each oligonucleotide included in an oligonucleotide set, but it is difficult to combine the specificity analysis results of the oligonucleotide set in consideration of probe-hybridized amplicons.

A conventional method of analyzing coverages in the unit of an amplicon, which is amplified by a primer pair, had problems in that match or mismatch patterns for all the sequences hit by the primer pair could not be shown; the specificity analysis results of probes for amplicons could not be obtained; and specificity analysis could not be performed when a plurality of oligonucleotides are present for each type of oligonucleotides.

(b) Unlike the conventional method, the present invention provides nucleic acid sequences with the generation of probe-hybridized amplicons and/or nucleic acid sequences without the generation of probe-hybridized amplicons by a combination of a forward primer, a probe, and a reverse primer included in an oligonucleotide set according to match or mismatch information and position information thereof, and thus can provide a coverage of the oligonucleotide set for a plurality of nucleic acid sequences, can analyze specificity of the oligonucleotide set (e.g., a primer pair and a probe), and can modify the sequences of oligonucleotides included in the oligonucleotide set for the improvement in specificity.

(c) Furthermore, according to the present invention, the specificity analysis results can be compared between an oligonucleotide set of an existing product and an oligonucleotide set of a new product, and the specificity change of the oligonucleotide set can be easily monitored.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1: Analysis of Specificity of Oligonucleotide Sets for Dermatophyte Diagnosis

By running a sequence coverage tool (SCT) providing the specificity analysis results of oligonucleotide sets (OSs), the specificity of oligonucleotide sets for Dermatophyte diagnosis was analyzed.

### 1. Input of Sequences of Oligonucleotide Sets

Sequence information of the oligonucleotide sets for Dermatophyte diagnosis was obtained from the paper (G. J. Wisselink. et al., Journal of Microbiological Methods, (2011)), and summarized in Table 1 below.

TABLE 1

| oligo | | | | |
|---|---|---|---|---|
| set | oligo name | oligo type | oligo sequence (5′ to 3′) | Note |
| 11 | Rubviol-FW | Forward primer | CTGCCAGGGAGAGCCGT | SEQ ID NO: 1 |
| | Rub-MGB | Probe 1 | CACCAAGAAAAAATTCTCTGAA | SEQ ID NO: 2 |
| | Viol-MGB | Probe 2 | CACCAAGGAAAATTCTCTGAA | SEQ ID NO: 3 |
| | Rubviol-REV | Reverse primer | GCTTGCTAAACGCTCAGACTGA | SEQ ID NO: 4 |
| 22 | Tons_FW | Forward primer | CTCTCCCAGGAGAGCCGTT | SEQ ID NO: 5 |
| | Tons_MGB | Probe | TTGAGCCGCTATAAAG | SEQ ID NO: 6 |
| | Tons_REV | Reverse primer | TGCTTGCTAACGCTCAGACTGA | SEQ ID NO: 7 |

The oligo in Table 1 above and the following table represents an oligonucleotide.

The sequences of the oligonucleotide sets were input through a user interface (UI) window for the SCT program according to each of the oligonucleotide sets.

The paper describes that the primer pair, probe 1, and probe 2 of the oligonucleotide set 1 target Trichophyton rubrum and Trichophyton violaceum, Trichophyton rubrum, and Trichophyton violaceum, respectively, and the oligonucleotide set 2 targets Trichophyton tonsurans.

### 2. Creation of Nucleotide Database Containing Nucleic Acid Sequences of Taxonomy Trichophyton and Database Selection

For analysis of specificity of the oligonucleotide sets, a nucleotide database including a plurality of nucleic acid sequences corresponding to the taxonomy name Trichophyton (Taxonomy ID: 5550) was created. In the nucleotide database, sequences of nucleic acid sequences for taxonomy belonging to the subclass of Trichophyton were collected. The nucleotide database is created to include a plurality of nucleic acid sequences corresponding to the Taxonomy name Trichophyton (Taxonomy ID: 5550) and its subclass, from the nucleotide database downloaded from the nucleotide databases in GenBank.

In addition, the created nucleotide database was selected through the UI window.

### 3. SCT Program Running

Through the UI window, the sequences of the oligonucleotide sets were input, the nucleotide database was selected, and then SCT program was run.

The SCT program was run to proceed in the following order: (1) BLAST was performed on each of the nucleic acid sequences contained in the nucleotide database by using each of the oligonucleotides included in the oligonucleotide sets as a query sequence, thereby providing match or mismatch information (number of mismatches (match or mismatch type) or mismatch pattern) and position information of each oligonucleotide. (2) The oligonucleotides were combined in the order of a forward primer and a probe, a probe and a reverse primer, or a forward primer, a probe, and a reverse primer, to generate probe-hybridized amplicons. (3) In a case where a plurality of probe-hybridized amplicons were formed for one nucleic acid sequence, a main probe-hybridized amplicon was selected therefrom, and a probe-hybridized amplicon generated by a combination of oligonucleotides, which had further three mismatches in addition to the sum of the numbers of mismatches by the combination of oligonucleotides generating the main probe-hybridized amplicon, was selected as a candidate probe-hybridized amplicon. (4) The sequences having mismatch patterns were grouped according to sequence identity for each type of oligonucleotides included in a combination of oligonucleotides generating probe-hybridized amplicons, and the information on mismatch patterns of each combination of oligonucleotides having the sequence with the grouped mismatch patterns and generating probe-hybridized amplicons was provided. (5) Nucleic acid sequences with the occurrence(generation) of probe-hybridized amplicon matches, nucleic acid sequences with the occurrence(generation) of probe-hybridized amplicon mismatches, and nucleic acid sequences without the generation of probe-hybridized amplicons (probe-hybridized amplicon fail nucleic acid sequences) were classified for each oligonucleotide set.

The classified nucleic acid sequences with the occurrence of probe-hybridized amplicon matches indicate that at least one probe-hybridized amplicon is generated, probe-hybridized amplicons generated by a combination of all matched oligonucleotides are generated, and the length range of the probe-hybridized amplicons falls within ±50% of the predetermined probe-hybridized amplicon length. The classified nucleic acid sequences with the occurrence of probe-hybridized amplicon mismatches indicate that at least one probe-hybridized amplicon is generated, the number of mismatches of at least one oligonucleotide included in a combination of oligonucleotides generating the at least one probe-hybridized amplicon is less than 5 and the length of the probe-hybridized amplicons exceeds ±50% of the predetermined probe-hybridized amplicon length.

Meanwhile, the nucleic acid sequences without the generation of probe-hybridized amplicons (probe-hybridized amplicon fail nucleic acid sequences) include: nucleic acid sequences with respect to oligonucleotides not generating probe-hybridized amplicons; nucleic acid sequences with respect to a combination of oligonucleotides generating probe-hybridized amplicons with a length exceeding 1,500 bp; and nucleic acid sequences with respect to a combination of oligonucleotides generating probe-hybridized amplicons wherein the number of mismatches of all of the oligonucleotides is 5 or more.

### 4. SCT Program Running Results

### (1) Analysis of Match or Mismatch Type of Each Oligonucleotide

TABLE 2

| | | | Number of nucleic acid sequences covered according to | | | | | |
|---|---|---|---|---|---|---|---|---|
| Oligo | | | number of mismatches (match or mismatch type) | | | | | |
| set | Oligo name | Oligo type | 0\|0 | 1\|0 | 2\|0 | 3\|0 | 3\|3-7\|5 | Total |
| 1 | Rubviol-FW | Forward primer | 1014 | 29 | 8 | 19 | 895 | 1965 |
| | Rub-MGB | Probe 1 | 932 | 7 | 4 | 1 | 817 | 1761 |
| | Viol-MGB | Probe 2 | 143 | 1 | 2 | 1 | 430 | 577 |
| | Rubviol-REV | Reverse primer | 1055 | 670 | 172 | 0 | 20 | 1917 |
| 2 | Tons_FW | Forward primer | 471 | 515 | 30 | 177 | 638 | 1831 |
| | Tons_MGB | Probe | 382 | 1 | 3 | 570 | 884 | 1840 |
| | Tons_REV | Reverse primer | 1157 | 478 | 42 | 1 | 54 | 1732 |

As can be seen from Table 2, the number of nucleic acid sequences covered according to the number of mismatches in each of the oligonucleotides included in the oligonucleotide sets are shown. In Table 2, 0|0 representing the number of mismatches indicates a match type (no mismatch), and the mismatch types 1|0, 2|0, 3|0, and 3β-7|5 indicates 1, 2, 3, and 4-7 mismatches, respectively. The oligonucleotides included in oligonucleotide sets 1 and 2 are not DPO primers including a 5′-high Tm specificity portion, a separation portion, and a 3′-low Tm specificity portion, and thus the rear part on the basis of the bar in the match or mismatch type may be expressed or construed as N. Specifically, the match type 0|0 may be expressed or construed as 0|N, and the mismatch types 1|0, 2|0, 3|0 and 3β-7|5 may be expressed or construed as 1|N, 2|N, 3|N, and 3|N-7|N.

(2) Number of Nucleic Acid Sequences with the Generation of Probe-Hybridized Amplicons According to Match Type in Each Oligonucleotide Set

TABLE 3

| | | | Set 1 | | | | | | |
|---|---|---|---|---|---|---|---|---|---|
| Tax | Tax | | Rubviol- | Rub- | Viol- | Rubviol- | Set 2 | | |
| ID | name | Match type | FW | MGB | MGB | REV | Tons_FW | Tons_MGB | Tons_REV |
| All | | Amp(0\|0) | 982 | | | | 380 | | |
| | | Oligo type | 1014 | 1075 | | 1055 | 471 | 382 | 1157 |
| | | (0\|0) | | | | | | | |
| | | 0\|0 | 1014 | 932 | 143 | 1055 | 471 | 382 | 1157 |
| | | 1\|0 | 29 | 7 | 1 | 670 | 515 | 1 | 478 |
| | | 2\|0-3\|2 | 27 | 5 | 3 | 172 | 207 | 573 | 43 |
| | | -7\|5 | 895 | 817 | 430 | 20 | 638 | 884 | 54 |
| 5550 | Trichophyton | Amp(0\|0) | 982 | | | | 380 | | |
| | | Oligo type | 1014 | 1075 | | 1055 | 471 | 382 | 1157 |
| | | (0\|0) | | | | | | | |
| | | 0\|0 | 1014 | 932 | 143 | 1055 | 471 | 382 | 1157 |
| | | 1\|0 | 29 | 7 | 1 | 670 | 515 | 1 | 478 |
| | | 2\|0-3\|2 | 27 | 5 | 3 | 172 | 207 | 573 | 43 |
| | | -7\|5 | 895 | 817 | 430 | 20 | 638 | 884 | 54 |

In Table 2 above and the following table, Tax represents taxonomy, Set represents oligonucleotide set, and Amp represents probe-hybridized amplicon.

As can be seen from Table 2, the number of probe-hybridized amplicons can be obtained when all the oligonucleotides included in oligonucleotide set 1 are combined to have match types (0|0, i.e., 0|N). It is confirmed whether each of the oligonucleotides included in the oligonucleotide sets had the match type (0|N), but in a case where two or more oligonucleotides are present in the same oligonucleotide type, only one of the oligonucleotides needs to have the match type (0|N).

Table 2 shows a case where a nucleotide database containing nucleic acid sequences collected for the nucleic acid sequences of Trichophyton was provided. However, if sequences of oligonucleotide sets designed to amplify and detect Enterovirus A and Enterovirus B were input, a nucleotide database containing nucleic acid sequences of Enterovirus A (Tax ID 138948) and Enterovirus B (Tax ID 138949) was provided, and then the SCT was run, the number of nucleic acid sequences with the generation of probe-hybridized amplicons in a match type for each of Enterovirus A and Enterovirus B could be provided as shown in the lower part of Table 2. If the prevalence of Enterovirus A is higher than that of Enterovirus B, when the number of nucleic acid sequences with the generation of probe-hybridized amplicons by an oligonucleotide set in a match type was high for Enterovirus B, the oligonucleotide set may be determined as being wrong designed. These results can be used to make a such analysis.

### (3) Oligonucleotide Set Comparison

From the comparison results of oligonucleotide sets for 6619 nucleic acid sequences, the comparison results of oligonucleotide sets for 10 nucleic acid sequences are summarized in Table 4.

TABLE 4

| Accession number | Set 1 | Set 2 | Tax name | Tax ID |
|---|---|---|---|---|
| LC035415.1 | AmpMismatch | AmpMatch | T. tonsurans | 34387 |
| LC035428.1 | AmpMismatch | AmpMatch | T. tonsurans | 34387 |
| KT155691.1 | AmpMismatch | AmpMatch | T. tonsurans | 34387 |
| AF170460.1 | AmpMatch | Fail | T. rubrum | 5551 |
| AJ270792.1 | AmpMatch | Fail | T. rubrum | 5551 |
| MK298851.1 | AmpMatch | Fail | T. violaceum | 34388 |
| MH859437.1 | AmpMatch | Fail | T. rubrum | 5551 |
| MK298971.1 | AmpMatch | Fail | T. violaceum | 34388 |
| MK806596.1 | AmpMatch | Fail | T. violaceum | 34388 |
| KT285210.1 | Fail | AmpMismatch | T. metagrophytes | 523103 |

In Table 4, AmpMatch represents the probe-hybridizing amplicon match, indicating that an oligonucleotide set produces probe-hybridized amplicon matches for a nucleic acid sequence (sequence of accession number); AmpMismatch represents the probe-hybridized amplicon mismatch, indicating that an oligonucleotide set produces probe-hybridized amplicon mismatches for a nucleic acid sequence (sequence of accession number); and Fail indicates that an oligonucleotide set does not generate probe-hybridized amplicons for a nucleic acid sequence (sequence of accession number). However, Fail indicates not only that probe-hybridized amplicons are not generated, but also that though probe-hybridization amplicons are generated, the length of the amplicons exceeds 1500 bp or the number of mismatches of all the oligonucleotides included in a combination of oligonucleotides generating the probe-hybridized amplicons is 5 or more.

Through Table 4, for each of the plurality of nucleic acid sequence, nucleic acid sequences covered by oligonucleotide sets 1 and 2 could be compared with each other. If oligonucleotide set 1 is a conventionally designed oligonucleotide set (an existing product) and oligonucleotide set 2 is a recently designed oligonucleotide set (a new product), nucleic acid sequences covered by the existing product and the new product can be compared.

### (4) Classification of Nucleic Acid Sequences for Each Oligonucleotide Set

Nucleic acid sequences producing probe-hybridized amplicon matches, nucleic acid sequences producing probe-hybridized amplicon mismatches, and nucleic acid sequences without the generation of probe-hybridized amplicons were classified for each oligonucleotide set.

1) The Results by Oligonucleotide Set 1 were Classified as Below:

\textcircled{1} Probe-Hybridized Amplicon Match

Among 982 nucleic acid sequences producing probe-hybridized amplicon matches, 10 nucleic acid sequences were summarized in Table 5 below:

TABLE 5

| AmpSize: | | | | | | | | | | | | | |
|---|---|---|---|---|---|---|---|---|---|---|---|---|---|
| 121 bp | Rubviol-FW | | | Rub-MGB | | | Viol-MGB | | | Rubviol-REV | | | |
| Acc | M.T | S | E | M.T | S | E | M.T | S | E | M.T | S | E | Tax name |
| Z97993.1 | 0\|N | 182 | 198 | 0\|N | 253 | 274 | 2\|N | 253 | 274 | -0\|N | 281 | 302 | T. rubrum |
| U18352.1 | 0\|N | 182 | 198 | 0\|N | 253 | 274 | 2\|N | 253 | 274 | -0\|N | 281 | 302 | |
| MP224338.1 | 0\|N | 137 | 153 | 0\|N | 208 | 229 | 2\|N | 208 | 229 | -0\|N | 236 | 257 | |
| MN966527.1 | 0\|N | 160 | 176 | 0\|N | 231 | 252 | 2\|N | 231 | 252 | -0\|N | 259 | 280 | |
| MN893238.1 | 0\|N | 173 | 189 | 0\|N | 244 | 265 | 2\|N | 244 | 265 | -0\|N | 272 | 293 | |
| MN808790.1 | 0\|N | 160 | 176 | 2\|N | 231 | 251 | 0\|N | 231 | 251 | -0\|N | 258 | 279 | T. violaceum |
| MN807421.1 | 0\|N | 135 | 151 | 2\|N | 206 | 226 | 0\|N | 206 | 226 | -0\|N | 233 | 254 | |
| MN807372.1 | 0\|N | 137 | 153 | 2\|N | 208 | 228 | 0\|N | 208 | 228 | -0\|N | 235 | 256 | |
| MN737950.1 | 0\|N | 141 | 157 | 2\|N | 212 | 232 | 0\|N | 212 | 232 | -0\|N | 239 | 260 | |
| MN737949.1 | 0\|N | 141 | 157 | 2\|N | 212 | 232 | 0\|N | 212 | 232 | -0\|N | 239 | 260 | |

In Table 5 and the following table, AmpSize, Acc, M.T, S, and E represent the length of probe-hybridized amplicons, Accession No., match type, start point, and end point, respectively. The length of the probe-hybridized amplicons being 121 bp indicates a probe-hybridized amplicon length of highest frequency when oligonucleotides included in an oligonucleotide set are all combined in a match type to generate probe-hybridized amplicons.

Table 5 shows that for Accession Nos. Z97993.1 to MN893238.1, main probe-hybridized amplicons were generated by combinations of Rubviol-FW, Rub-MGB, and Rubviol-REV and candidate probe-hybridized amplicons were generated by combinations of Rubviol-FW, Viol-MGB, and Rubviol-REV. In addition, for Accession Nos. MN808790.1 to MN737949.1, main probe-hybridized amplicons were generated by combinations of Rubviol-FW, Viol-MGB, and Rubviol-REV and candidate probe-hybridized amplicons were generated by combinations of Rubviol-FW, Rub-MGB, and Rubviol-REV.

It could be verified from Table 5 that as disclosed in the paper, the primer pair and the probe Rub-MGB can cover T. rubrum by probe-hybridized amplicon matches and the primer pair and the probe Viol-MGB can cover T. violaceum by probe-hybridized amplicon matches.

Furthermore, among 982 nucleic acid sequences producing the probe-hybridized amplicon matches, 868 nucleic acid sequences were covered by the primer pair and the probe Rub-MGB, and the identification results of their specific taxonomy were as follows: Trichophyton rubrum (788), Trichophyton soudanense (64), Trichophyton mentagrophytes (5), Trichophyton violaceum (3), Trichophyton gourvilll (2), Trichophyton kuryangei (1), Trichophyton interdigitale (1), Trichophyton sp. (1), Trichophyton sp. SIN38 (1), Trichophyton rubrum var. flavum (1), Trichophyton circonvolutum (1).

Among 982 nucleic acid sequences producing the probe-hybridized amplicon matches, 114 nucleic acid sequences were covered by the primer pair and the probe Viol-MGB, and the identification results of their specific taxonomy were as follows: Trichophyton violaceum (114).

Through these results, information on the nucleic acid sequences covered by the combinations of the oligonucleotides included in oligonucleotide set 1 could be obtained.

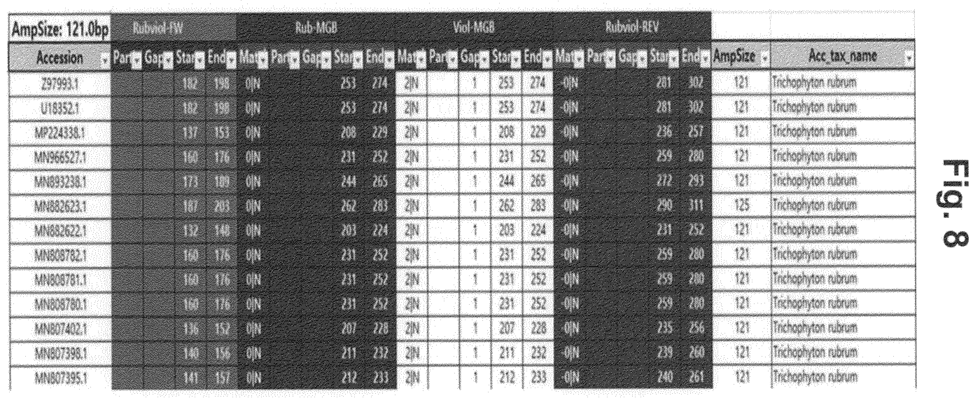

FIG. 8 shows the results in Excel file format for some of 982 nucleic acid sequences producing probe-hybridized amplicon matches (probe-hybridized amplicon match nucleic acid sequences), and Table 5 shows the summary of the results for the accession numbers listed in Table 5 from the results in the Excel file format. In FIG. 8, as for the nucleic acid sequences of the accession numbers, all the combinations of darkly shaded oligonucleotides have a match type and represent combinations generating main probe-hybridization amplicons, and the unshaded oligonucleotides having a mismatch type are candidate oligonucleotides used to generate candidate probe-hybridized amplicons. In FIG. 8, the dark grades for the oligonucleotide types, that is, the forward primer, the probe, and the reverse primer are dark orange, dark green and dark brown, respectively, and through such color differentiation, the combinations of oligonucleotides and nucleic acid sequences with the generation of probe-hybridized amplicons in a match type can be easily identified.

\textcircled{2} Probe-Hybridized Amplicon Mismatch

Among 988 nucleic acid sequences producing probe-hybridized amplicon matches, 10 nucleic acid sequences were summarized in Table 6 below:

TABLE 6

| AmpSize: | | | | | | | | | | | | | |
|---|---|---|---|---|---|---|---|---|---|---|---|---|---|
| 121 bp | Rubviol-FW | | | Rub-MGB | | | Viol-MGB | | | Rubviol-REV | | | |
| Acc | M.T | S | E | M.T | S | E | M.T | S | E | M.T | S | E | Tax name |
| MN306538.1 | 0\|N | 162 | 178 | 0\|N | 233 | 254 | 2\|N | 233 | 254 | -1\|N | 261 | 283 | T. rubrum |
| MF402917.1 | 0\|N | 160 | 176 | 0\|N | 231 | 252 | 2\|N | 231 | 252 | -1\|N | 259 | 280 | T. violaceum |
| AJ222626.1 | 0\|N | 131 | 147 | 0\|N | 202 | 223 | 2\|N | 202 | 223 | -2\|N | 230 | 251 | T. soudanense |
| MN885450.1 | 0\|N | 159 | 175 | 1\|N | 234 | 255 | 3\|N | 234 | 255 | -0\|N | 262 | 283 | T. sp. |
| MN882624.1 | 0\|N | 159 | 175 | 1\|N | 234 | 255 | 3\|N | 234 | 255 | -0\|N | 262 | 283 | T. rubrum |
| MF800876.1 | 0\|N | 108 | 124 | 1\|N | 179 | 200 | — | — | — | -0\|N | 207 | 228 | T. rubrum |
| KM822829.1 | 0\|N | 190 | 206 | 2\|N | 261 | 281 | 2\|N | 261 | 281 | -1\|N | 288 | 309 | T. rubrum |
| MN737880.1 | 0\|N | 141 | 157 | 2\|N | 212 | 232 | 0\|N | 212 | 232 | -1\|N | 239 | 260 | T. violaceum |
| MK806614.1 | 0\|N | 104 | 120 | 2\|N | 175 | 195 | 0\|N | 175 | 195 | -1\|N | 202 | 223 | T. violaceum |
| MK806613.1 | 0\|N | 104 | 120 | 2\|N | 175 | 195 | 0\|N | 175 | 195 | -1\|N | 202 | 223 | T. violaceum |

It can be seen from Table 6 that the reverse primer Rubviol-REV for Accession No. MN306538.1 has a mismatch type of −11 N, and thus a degenerate base needs to be introduced into Rubviol-REV in order to cover Accession No. MN306538.1 by a combination of Rubviol-FW, Rub-MGB, and Rubviol-REV. It can also be seen that in order to cover MN882624.1 and MF800876.1 by a combination of Rubviol-FW, Rub-MGB, and Rubviol-REV, a degenerate base needs to be introduced into the probe Rub-MGB since Rub-MGB has a mismatch type of 11N. It can also be seen that in order to cover MN737880.1, MK806614.1, and MK806613.1 by a combination of Rubviol-FW, Viol-MGB, and Rubviol-REV, a degenerate base needs to be introduced into the reverse primer Rubviol-REV since the reverse primer Rubviol-REV has a mismatch type of 11N.

As described above, it could be confirmed that the modification of oligonucleotides included in an oligonucleotide set can be achieved from the information of nucleic acid sequences with the occurrence of probe-hybridized amplicon mismatches.

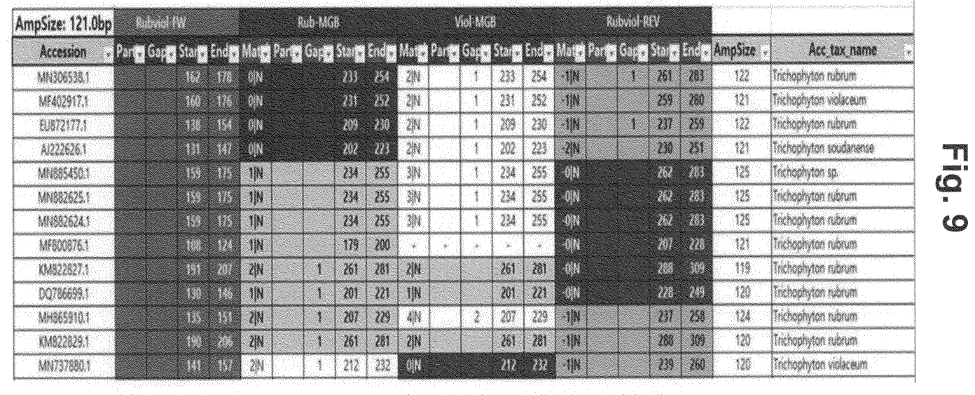

FIG. 9 shows the results in Excel file format for some of 988 nucleic acid sequences producing probe-hybridized amplicon mismatches (probe-hybridized amplicon mismatch nucleic acid sequences), and Table 6 shows the summary of the results for only the accession numbers listed in Table 6 from the results in the Excel file format. In FIG. 9, as for the nucleic acid sequences of the accession numbers, the darkly shaded oligonucleotides indicate having match types and the lightly shaded oligonucleotides indicate having mismatch types. In FIG. 9, the shades of the oligonucleotide types, that is, the forward primer, the probe, and the reverse primer are orange, green and brown, respectively, and dark colors were used for a match type and light colors were used for a mismatch type. Through such color differentiation, the combinations of oligonucleotides and nucleic acid sequences with the generation of probe-hybridized amplicon mismatches can be easily identified.

\textcircled{3} Probe-Hybridized Amplicon Fail

Among 22786 nucleic acid sequences without the generation of probe-hybridized amplicons, 10 nucleic acid sequences were summarized in Table 7 below:

TABLE 7

| AmpSize: 121 bp | Rubviol-FW | | | Rub-MGB | | | Viol-MGB | | | Rubviol-REV | | | |
|---|---|---|---|---|---|---|---|---|---|---|---|---|---|
| Acc | M.T | S | E | M.T | S | E | M.T | S | E | M.T | S | E | Tax name |
| QQSR01000170.1 | 5\|N | 16548 | 16564 | 7\|N | 16649 | 16670 | — | — | — | -7\|N | 16791 | 16812 | T. mentagrophytes |
| QQSR01000106.1 | -7\|N | 41908 | 41924 | 7\|N | 41851 | 41872 | — | — | — | 7\|N | 41795 | 41816 | T. mentagrophytes |
| Z98009.1 | 6\|N | 149 | 165 | 7\|N | 588 | 608 | — | — | — | — | — | — | T. equinum |
| QQSR01001364.1 | -7\|N | 193 | 209 | 7\|N | 57 | 78 | — | — | — | — | — | — | T. mentagrophytes. |
| QQSR01000593.1 | 6\|N | 1588 | 1604 | 7\|N | 1638 | 1659 | — | — | — | — | — | — | T. mentagrophytes. |

In Tables 6 and 7, the result for the match type and the start point and end point expressed as “-” indicates no hit, which largely encompasses four cases: 1) there is no sequence in the region of a nucleic acid sequence, corresponding to an oligonucleotide, that is, the nucleic acid sequence is a template partial; 2) there are too many mismatches in the region of a nucleic acid sequence, corresponding to an oligonucleotide, leading to the Blast no hit result; 3) the number of mismatches between an oligonucleotide and a nucleic acid sequence exceeds 7 (mismatch type: 7|N) or 12 (mismatch type: 7|5); and 4) there are a junk DNA base (i.e., N base) and a gap in the region of a nucleic acid sequence, corresponding to an oligonucleotide.

It can be seen whether the no hit expressed as “-” corresponds to one of the above four cases, by identifying information of nucleic acid sequence separately linked to Tables 6 and 7 (now shown in Tables 6 and 7).

The probe-hybridization amplicon fail nucleic acid sequences include: nucleic acid sequences without the generation of probe-hybridized amplicons by an oligonucleotide set for nucleic acid sequences (sequences of accession numbers); nucleic acid sequences, even if generating probe-hybridized amplicons, of which the length exceeds 1500 bp; and nucleic acid sequences wherein the number of mismatches in all the oligonucleotides included in a combination for generating the probe-hybridized amplicons is 5 or more.

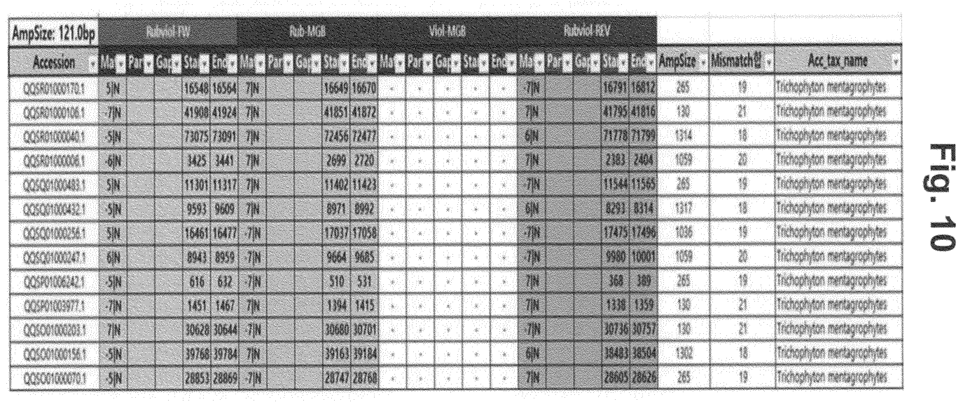

FIG. 10 shows the results in Excel file format for some of 22786 nucleic acid sequences without the generation of probe-hybridized amplicons (probe-hybridized amplicon fail nucleic acid sequences), and Table 7 shows the summary of the results for only the accession numbers listed in Table 7 from the results in the Excel file format. In FIG. 10, as for the nucleic acid sequences of the accession numbers, the lightly shaded oligonucleotides indicate having mismatch types. In FIG. 10, the light shades of the oligonucleotide types, that is, the forward primer, the probe, and the reverse primer are orange, green, and brown, respectively, and through such color differentiation, the nucleic acid sequences without the generation of probe-hybridized amplicons can be easily identified.

2) The Results by Oligonucleotide Set 2 were Classified as Below:

\textcircled{1} Probe-Hybridized Amplicon Match

Of the results for 380 nucleic acid sequences with the occurrence of probe-hybridized amplicon matches, the results for 3 nucleic acid sequences were summarized in Table 8 below:

TABLE 8

| AmpSize: 126 bp | Tons-FW | | | Tons-MGB | | | Tons-REV | | | |
|---|---|---|---|---|---|---|---|---|---|---|
| Acc | M.T | S | E | M.T | S | E | M.T | S | E | Tax name |
| Z98008.1 | 0\|N | 173 | 191 | -0\|N | 204 | 219 | -0\|N | 277 | 298 | T. tonsurans |
| MN966493.1 | 0\|N | 153 | 171 | -0\|N | 184 | 199 | -0\|N | 257 | 278 | T. tonsurans |
| MN950431.1 | 0\|N | 106 | 124 | -0\|N | 137 | 152 | -0\|N | 210 | 231 | T. tonsurans |

\textcircled{2} Probe-Hybridized Amplicon Mismatch

Of the results for 1460 nucleic acid sequences with the occurrence of probe-hybridized amplicon matches, the results for 3 nucleic acid sequences were summarized in Table 9 below:

TABLE 9

| AmpSize: 126 bp | Tons-FW | | | Tons-MGB | | | Tons-REV | | | |
|---|---|---|---|---|---|---|---|---|---|---|
| Acc | M.T | S | E | M.T | S | E | M.T | S | E | Tax name |
| MN950432.1 | 0\|N | 93 | 111 | -0\|N | 124 | 139 | -1\|N | 197 | 218 | T. tonsurans |
| MN807409.1 | 0\|N | 137 | 155 | -0\|N | 168 | 183 | -1\|N | 241 | 262 | T. tonsurans |
| Z98018.1 | 0\|N | 175 | 193 | -5\|N | 204 | 219 | -0\|N | 276 | 297 | T. simii |

\textcircled{3} Probe-Hybridized Amplicon Fail

Of the results for 24739 nucleic acid sequences without the occurrence of probe-hybridized amplicon matches, the results for 3 nucleic acid sequences were summarized in Table 10 below:

TABLE 10

| AmpSize: 126 bp | Tons-FW | | | Tons-MGB | | | Tons-REV | | | |
|---|---|---|---|---|---|---|---|---|---|---|
| Acc | M.T | S | E | M.T | S | E | M.T | S | E | Tax name |
| QQSR01000576.1 | 6\|N | 1049 | 1067 | 6\|N | 1351 | 1366 | -7\|N | 2460 | 2481 | T. mentagrophytes |
| QQSR01000270.1 | -7\|N | 5964 | 5982 | -6\|N | 5892 | 5907 | 7\|N | 4989 | 5010 | T. mentagrophytes |
| QQSR01000210.1 | -6\|N | 15138 | 15157 | -7\|N | 14396 | 14411 | 7\|N | 14290 | 14310 | T. mentagrophytes |

### (5) Providing Information of Mismatch Patterns of Combination of Oligonucleotides Generating Probe-Hybridized Amplicons in Oligonucleotide Set 1

FIGS. 11A and 11B show the information of the top two mismatch pattern information with a larger number of nucleic acid sequences in the mismatch pattern information of a combination of oligonucleotides generating probe-hybridized amplicons in oligonucleotide set 1. A single drawing is divided and shown in FIGS. 11A and 11B.

In FIGS. 11A and 11B, #Acc represents the number of accession numbers; #TP represents the number of partial templates or template partials; FpriName represents the forward primer name; ProbeName represents the probe name; RpriName represents the reverse primer name; Fpri_MT, Probe_MT, and Rpri_MT represent match or mismatch types of the forward primer, probe, and reverse primer, respectively; Fpri_PT, Probe_PT, and Rpri_PT represent information of mismatch patterns of the forward primer, probe and reverse primer, respectively; TaxName represents the taxonomy name; and Acclist represents the accession number list.

In FIGS. 11A and 11B, “-” indicates a partial template or a template partial, and the accession number list can be confirmed by clicking the link.

As can be confirmed from FIGS. 11A and 11B, the sequences having mismatch patterns are grouped according to sequence identity for each oligonucleotide type of oligonucleotide set 1, and the information of mismatch patterns of a combination of oligonucleotides having the sequence with the grouped mismatch patterns and generating probe-hybridized amplicons could be identified.

It could be confirmed through the information of mismatch patterns in FIGS. 11A and 11B that the coverage of a set of oligonucleotides can be improved by modifying the oligonucleotides in the unit of a combination of oligonucleotides generating probe-hybridized amplicons.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A computer-implemented method for providing a coverage of an oligonucleotide set for a plurality of nucleic acid sequences, the method comprising:
(a) inputting sequences of an oligonucleotide set, wherein the oligonucleotide set includes a primer pair and a probe as oligonucleotides;
(b) providing a nucleotide database, wherein the nucleotide database contains a plurality of nucleic acid sequences;
(c) providing match or mismatch information and position information of each of the oligonucleotides included in the oligonucleotide set for each of the plurality of nucleic acid sequences by confirming whether the sequences of the oligonucleotide set are mismatched to the plurality of nucleic acid sequences contained in the nucleotide database, wherein the match or mismatch information indicates the number of matches or mismatches and/or a mismatch pattern of each of the oligonucleotides included in the oligonucleotide set for each of the plurality of nucleic acid sequences;
(d) confirming whether probe-hybridized amplicons are generated by the oligonucleotide set for each of the plurality of nucleic acid sequences, wherein the primer pair includes a forward primer and a reverse primer; the probe-hybridized amplicons are products amplified by the forward primer and/or reverse primer and indicate amplicons detected by hybridization of the probe included in the oligonucleotide set; and at least one of the probe-hybridized amplicons is formed by a combination of the oligonucleotides according to the match or mismatch information and position information of each of the oligonucleotides included in the oligonucleotide set for each of the plurality of nucleic acid sequences; and
(e) providing nucleic acid sequences with the generation of probe-hybridized amplicons by the oligonucleotide set and/or nucleic acid sequences without the generation of probe-hybridized amplicons by the oligonucleotide set, wherein the nucleic acid sequences with the generation of probe-hybridized amplicons are covered by the oligonucleotide set and the nucleic acid sequences without the generation of probe-hybridized amplicons are not covered by the oligonucleotide set.

2. The method according to claim 1, wherein the oligonucleotide set in step (a) further comprises at least one oligonucleotide selected from the oligonucleotides consisting of at least one forward primer, at least one probe, and at least one reverse primer.

3. The method according to claim 1, wherein the nucleotide database in step (b) is a nucleotide database containing nucleic acid sequences collected by an identifier selected from identifiers composed of taxonomy ID, taxonomy name, organism name, and target nucleic acid molecule name, from a public-accessible nucleotide database or a nucleotide database obtaining by downloading the public-accessible nucleotide database, or a nucleotide database containing nucleic acid sequences collected by a user.

4. The method according to claim 3, wherein the public-accessible nucleotide database is a nucleotide database selected from the group consisting of GenBank, European Molecular Biology Laboratory (EMBL), and DNA DataBank of Japan (DDBJ).

5. The method according to claim 1, wherein the plurality of nucleic acid sequences in step (b) include a plurality of target nucleic acid sequences and/or a plurality of non-target nucleic acid sequences.

6. The method according to claim 1, wherein the probe-hybridized amplicons in step (d) are generated in the order of the forward primer and the probe, the probe and the reverse primer, or the forward primer, the probe and the reverse primer.

7. The method according to claim 1, wherein the probe-hybridized amplicons in step (d) are generated or selected to satisfy at least one of the following criteria:
(i) a probe-hybridized amplicon length being less than a predetermined value, wherein the length indicates a length from a nucleotide at the 5′-end of a forward and/or reverse primer to a nucleotide at the 3′-end of an amplicon amplified by the forward and/or reverse primer; and
(ii) the number of mismatches in each of oligonucleotides included in a combination of oligonucleotides being less than a predetermined value.

8. The method according to claim 1, wherein the method further comprises, after step (d), d-1) selecting, as a main probe-hybridized amplicon, a probe-hybridized amplicon satisfying at least one of selection criteria considering the following priorities, from the at least one formed probe-hybridized amplicon:
(i) a ratio of the sum of the number of mismatches and the number of partial nucleotides in oligonucleotides included in a combination of oligonucleotides relative to the number of the oligonucleotides included in the combination of oligonucleotides, wherein the oligonucleotides included in the combination of oligonucleotides include a probe and a forward primer and/or a reverse primer, and the lower the ratio, the higher the priority;
(ii) the number of mismatches in a probe included in a combination of oligonucleotides, wherein the smaller the number, the higher the priority;
(iii) a ratio of the number of mismatches in a region from the 3′-end of a primer to a nucleotide spaced apart from the 3′-end of the primer by a predetermined length relative to the number of primers included in a combination of oligonucleotides, wherein the lower the ratio, the higher the priority; and
(iv) the number of mismatches in a primer included in a combination of oligonucleotides, wherein the smaller the number, the higher the priority.

9. The method according to claim 1, wherein the method further comprises, after step (d), the following steps:
d-2) grouping, according to sequence identity, sequences having a mismatch pattern for each of types of oligonucleotides included in the combination of the oligonucleotides, wherein the grouped sequences having a mismatch pattern has a mismatch pattern having the same mismatch position between the oligonucleotide and the nucleic acid sequence in oligonucleotides of the same type having the mismatch pattern and has a mismatch pattern having the same base between oligonucleotide sequences and between nucleic acid sequences at the mismatch position; and the oligonucleotide type indicates a type of oligonucleotides as a forward primer, a probe, and a reverse primer; and
d-3) providing information on the mismatch pattern for each combination of oligonucleotides having the same mismatch pattern and generating probe-hybridized amplicons by combining oligonucleotides having the grouped sequences having a mismatch pattern, wherein the information on the mismatch pattern indicates oligonucleotide sequences and nucleic acid sequences having the mismatch pattern, the number of nucleic acid sequences having the mismatch pattern, or a list of identifiers.

10. The method according to claim 1, wherein the nucleic acid sequences with the generation of probe-hybridized amplicons by the oligonucleotide set in step (e) include nucleic acid sequences satisfying the following criteria (i) and (ii) and nucleic acid sequences satisfying the following criteria (iii) and (iv):
(i) a predetermined probe-hybridized amplicon length range;
(ii) the number of mismatches for nucleic acid sequences in all the oligonucleotides included in a combination of the oligonucleotides being 0 (zero);
(iii) exceeding the length range of (i); and
(iv) the number of mismatches in at least one oligonucleotide of the oligonucleotides included in a combination of the oligonucleotides being less than a predetermined value.

11. The method according to claim 10, wherein the predetermined probe-hybridized amplicon length range of (i) is determined by a user or determined by a probe-hybridized amplicon length of high frequency among the lengths of the probe-hybridized amplicons generated by a combination of oligonucleotides having no mismatches for a plurality of nucleic acid sequences.

12. The method according to claim 1, wherein the nucleic acid sequences in step (e) contain information on nucleic acid sequences selected from the group consisting of the number of nucleic acid sequences, accession numbers of the nucleic acid sequences (Accession Nos.), taxonomy names to which the nucleic acid sequences belong, taxonomy IDs assigned to the taxonomy names, ratios of nucleic acid sequences covered by combinations of the oligonucleotides relative to the total nucleic acid sequences, and mismatch patterns of oligonucleotides included in the combination of the oligonucleotides.

13. The method according to claim 1, wherein, when the plurality of nucleic acid sequences contained in the nucleotide database of step (b) are a plurality of target nucleic acid sequences, step (e) provides nucleic acid sequences with the generation of probe-hybridized amplicons by the oligonucleotide set, thereby providing a plurality of target nucleic acid sequences covered by the oligonucleotide set.

14. The method according to claim 1, wherein the plurality of nucleic acid sequences contained in the nucleotide database of step (b) are a plurality of non-target nucleic acid sequences, and step (e) provides information on nucleic acid sequences without the generation of probe-hybridized amplicons by the oligonucleotide set, thereby providing a plurality of non-target nucleic acid sequences not covered by the oligonucleotide set.

15. The method according to claim 1, wherein the method further comprises, after step (a), a-1) inputting sequences of at least one oligonucleotide set, which are different from the sequences of the oligonucleotide set in step (a).

16. The method according to claim 15, wherein, the at least one oligonucleotide set in step a-1) is the same as or different from the oligonucleotide set in step (a) in view of a nucleic acid molecule or organism to be covered.

17. The method according to claim 15, wherein the sequence of at least one oligonucleotide of the oligonucleotides included in the at least one oligonucleotide set in step a-1) is different from the sequences of the oligonucleotides included in the oligonucleotide set in step (a).

18. The method according to claim 15, wherein the method further comprises, f) comparing the coverage for a plurality of nucleic acid sequences by the oligonucleotide set in step (a) with the coverage for a plurality of nucleic acid sequences by at least one oligonucleotide set in step a-1).

19. The method according to claim 18, wherein the oligonucleotide set in step (a) and the at least one oligonucleotide set in step a-1) are oligonucleotide sets designed at different time points.

20. The method according to claim 1, wherein the method further comprises the following steps:
(f) performing steps (a) to (e) on a nucleotide database provided at a time point different from that in step (b); and
(g) comparing the resultant in step (e) and the resultant in step (e) of step (f).

21. A computer readable storage medium comprising instructions to configure a processor to perform a method for providing a coverage of an oligonucleotide set for a plurality of nucleic acid sequences, the method comprising:
(a) inputting sequences of an oligonucleotide set, wherein the oligonucleotide set includes a primer pair and a probe as oligonucleotides;
(b) providing a nucleotide database, wherein the nucleotide database contains a plurality of nucleic acid sequences;
(c) providing match or mismatch information and position information of each of the oligonucleotides included in the oligonucleotide set for each of the plurality of nucleic acid sequences by confirming whether the sequences of the oligonucleotide set are mismatched to the plurality of nucleic acid sequences contained in the nucleotide database, wherein the match or mismatch information indicates the number of matches or mismatches and/or a mismatch pattern of each of the oligonucleotides included in the oligonucleotide set for each of the plurality of nucleic acid sequences;
(d) confirming whether probe-hybridized amplicons are generated by the oligonucleotide set for each of the plurality of nucleic acid sequences, wherein the primer pair includes a forward primer and a reverse primer; the probe-hybridized amplicons are products amplified by the forward primer and/or reverse primer and indicate amplicons detected by hybridization of the probe included in the oligonucleotide set; and at least one of the probe-hybridized amplicons is formed by a combination of the oligonucleotides according to the match or mismatch information and position information of each of the oligonucleotides included in the oligonucleotide set for each of the plurality of nucleic acid sequences; and
(e) providing nucleic acid sequences with the generation of probe-hybridized amplicons by the oligonucleotide set and/or nucleic acid sequences without the generation of probe-hybridized amplicons by the oligonucleotide set, wherein the nucleic acid sequences with the generation of probe-hybridized amplicons are covered by the oligonucleotide set and the nucleic acid sequences without the generation of probe-hybridized amplicons are not covered by the oligonucleotide set.


SEQUENCE LISTING (7 sequences)

SEQ ID NO: 1 - DNA, 17 bp, Artificial Sequence
  Synthetic nucleotide_Rubviol-FW
ctgccagggagagccgt

SEQ ID NO: 2 - DNA, 22 bp, Artificial Sequence
  Synthetic nucleotide_Rub-MGB
caccaagaaaaaattctctgaa

SEQ ID NO: 3 - DNA, 21 bp, Artificial Sequence
  Synthetic nucleotide_Viol-MGB
caccaaggaaaattctctgaa

SEQ ID NO: 4 - DNA, 22 bp, Artificial Sequence
  Synthetic nucleotide_Rubviol-REV
gcttgctaaacgctcagactga

SEQ ID NO: 5 - DNA, 19 bp, Artificial Sequence
  Synthetic nucleotide_Tons_FW
ctctcccaggagagccgtt

SEQ ID NO: 6 - DNA, 16 bp, Artificial Sequence
  Synthetic nucleotide_Tons_MGB
ttgagccgctataaag

SEQ ID NO: 7 - DNA, 22 bp, Artificial Sequence
  Synthetic nucleotide_Tons_REV
tgcttgctaacgctcagactga